(19) United States
(12) Patent Application Publication
(10) Pub. No.: US 2021/0299273 A1
(43) Pub. Date: Sep. 30, 2021

(54) NANOPARTICLE COMPOSITIONS

(71) Applicant: January Therapeutics, Inc., San Diego, CA (US)

(72) Inventors: Raj RAHEJA, San Diego, CA (US); Robin M. JACKMAN, San Diego, CA (US); Jason A. KAHANA, San Diego, CA (US)

(21) Appl. No.: 17/262,151

(22) PCT Filed: Jul. 23, 2019
(86) PCT No.: PCT/US19/43109

§ 371 Date: Jan. 21, 2021

Related U.S. Application Data
(60) Provisional application No. 62/702,835, filed on Jul. 24, 2018.

(51) Int. Cl. A61K 47/69; A61K 47/54; A61K 47/55; A61K 9/19; A61K 47/64; A61K 9/51

(52) U.S. Cl. CPC A61K 47/6929; A61K 47/545; A61K 47/55; B82Y 5/00; A61K 47/643; A61K 9/5192; A61K 9/19

(57) ABSTRACT

Provided herein are nanoparticle compositions comprising a pharmaceutically acceptable carrier and a compound of Formula (I): A-L-B.

### CROSS-REFERENCE

This application claims benefit of U.S. Provisional Application No. 62/702,835, filed on Jul. 24, 2018, which is herein incorporated by reference in its entirety.

### BACKGROUND

In recent years, new classes of heterobifunctional molecules, also known as proteolysis targeting chimeras (PROTACs), have emerged comprising a compound that binds to a target protein and a compound that binds to an E3 ubiquitin ligase. The heterobifunctional compound simultaneously binds to the target protein and the E3 ubiquitin ligase, bringing both proteins in spatial proximity to induce ubiquitination, and thus marking the target protein for proteasome degradation.

### BRIEF SUMMARY OF THE DISCLOSURE

This disclosure provides, for example, nanoparticle compositions comprising compounds used to selectively induce the degradation of a target protein, their use as medicinal agents, and processes for their preparation. The disclosure also provides for the use of the nanoparticle compositions described herein as medicaments and/or in the manufacture of medicaments for the treatment of disease.

Provided in one aspect is a composition comprising nanoparticles, wherein the nanoparticles comprise a compound of Formula (I), or a pharmaceutically acceptable salt thereof, and a pharmaceutically acceptable carrier; wherein the pharmaceutically acceptable carrier comprises albumin and the compound of Formula (I) has the structure:

A-L-B Formula (I);

wherein:
A is a compound that binds to an E3 ubiquitin ligase;
L is a linker comprising at least two carbon atoms; and
B is a ligand which binds to a target protein or polypeptide which is to be mono-ubiquitinated or poly-ubiquitinated by the E3 ligase and thereby degraded, and is linked to the A group through the L group.

In some embodiments, A is selected from a cereblon binder, a Von Hippel-Lindau tumor suppressor protein (VHL) binder, an inhibitor of apoptosis protein (IAP) binder, a Kelch-like ECH-associated protein 1 (Keap1) binder, a mouse double minute 2 homolog (MDM2) binder, and beta-transducin repeat containing protein (b-TrCP) binder. In some embodiments, A is a cereblon binder. In some embodiments, A is a cereblon binder selected from lenalidomide, pomalidomide, and thalidomide. In some embodiments, A is a VHL binder. In some embodiments, A is an IAP binder. In some embodiments, A is an IAP binder selected from an X-linked inhibitor of apoptosis protein (XIAP), cellular inhibitor of apoptosis protein-1 (cIAP1), cellular inhibitor of apoptosis protein-2 (cIAP2), neuronal apoptosis inhibitory protein (NAIP), livin, and survivin. In some embodiments, A is a Keap1 binder. In some embodiments, A is an MDM2 binder. In some embodiments, A is a b-TrCP binder.

In some embodiments, the nanoparticles have an average diameter of about 1000 nm or less for at least about 15 minutes after nanoparticle formation. In some embodiments, the nanoparticles have an average diameter of about 10 nm or greater for at least about 15 minutes after nanoparticle formation. In some embodiments, the nanoparticles have an average diameter of from about 10 nm to about 1000 nm for at least about 15 minutes after nanoparticle formation.

In some embodiments, the nanoparticles have an average diameter of about 1000 nm or less for at least about 2 hours after nanoparticle formation. In some embodiments, the nanoparticles have an average diameter of about 10 nm or greater for at least about 2 hours nanoparticle formation. In some embodiments, the nanoparticles have an average diameter of from about 10 nm to about 1000 nm for at least about 2 hours after nanoparticle formation.

In some embodiments, the nanoparticles have an average diameter of from about 10 nm to about 1000 nm. In some embodiments, the nanoparticles have an average diameter of from about 30 nm to about 250 nm.

In some embodiments, the albumin is human serum albumin. In some embodiments, the molar ratio of the compound of Formula (I), or a pharmaceutically acceptable salt thereof, to pharmaceutically acceptable carrier is from about 1:1 to about 20:1. In some embodiments, the molar ratio of the compound of Formula (I), or a pharmaceutically acceptable salt thereof, to pharmaceutically acceptable carrier is from about 2:1 to about 12:1. In some embodiments, the nanoparticles are suspended, dissolved, or emulsified in a liquid. In some embodiments, the composition is sterile filterable.

In some embodiments, the composition is dehydrated. In some embodiments, the composition is a lyophilized composition. In some embodiments, the composition comprises from about 0.9% to about 24% by weight of the compound of Formula (I), or a pharmaceutically acceptable salt thereof. In some embodiments, the composition comprises from about 1.8% to about 16% by weight of the compound of Formula (I), or a pharmaceutically acceptable salt thereof. In some embodiments, the composition comprises from about 76% to about 99% by weight of the pharmaceutically acceptable carrier. In some embodiments, the composition comprises from about 84% to about 98% by weight of the pharmaceutically acceptable carrier.

In some embodiments, the composition is reconstituted with an appropriate biocompatible liquid to provide a reconstituted composition. In some embodiments, the appropriate biocompatible liquid is a buffered solution. In some embodiments, the appropriate biocompatible liquid is a solution comprising dextrose. In some embodiments, the appropriate biocompatible liquid is a solution comprising one or more salts. In some embodiments, the appropriate biocompatible liquid is sterile water, saline, phosphate-buffered saline, 5% dextrose in water solution, Ringer's solution, or Ringer's lactate solution. In some embodiments, the nanoparticles have an average diameter of from about 10 nm to about 1000 nm after reconstitution. In some embodiments, the nanoparticles have an average diameter of from about 30 nm to about 250 nm after reconstitution.

In some embodiments, the composition is suitable for injection. In some embodiments, the composition is suitable for intravenous administration. In some embodiments, the composition is administered intraperitoneally, intraarterially, intrapulmonarily, orally, by inhalation, intravesicularly, intramuscularly, intratracheally, subcutaneously, intraocularly, intrathecally, intratumorally, or transdermally.

Provided herein in another aspect is a method of treating a disease in a subject in need thereof comprising administering the composition comprising nanoparticles, wherein the nanoparticles comprise a compound of Formula (I), or a pharmaceutically acceptable salt thereof; and a pharmaceutically acceptable carrier; wherein the pharmaceutically acceptable carrier comprises albumin.

Provided in another aspect is a method of delivering a compound of Formula (I), or a pharmaceutically acceptable salt thereof, to a subject in need thereof comprising administering any one of the compositions described herein.

Provided in another aspect is a process of preparing any one of the compositions described herein comprising
a) dissolving a compound of Formula (I), or a pharmaceutically acceptable salt thereof, in a volatile solvent to form a solution comprising a compound of Formula (I), or a pharmaceutically acceptable salt thereof;
b) adding the solution comprising the dissolved compound of Formula (I), or a pharmaceutically acceptable salt thereof, to a pharmaceutically acceptable carrier in an aqueous solution to form an emulsion;
c) subjecting the emulsion to homogenization to form a homogenized emulsion; and
d) subjecting the homogenized emulsion to evaporation of the volatile solvent to form any one of the compositions described herein.

In some embodiments, the volatile solvent is a chlorinated solvent, alcohol, ketone, ester, ether, acetonitrile, or any combination thereof. In some embodiments, the volatile solvent is chloroform, ethanol, methanol, or butanol. In some embodiments, the homogenization is high pressure homogenization. In some embodiments, the emulsion is cycled through high pressure homogenization for an appropriate amount of cycles. In some embodiments, the appropriate amount of cycles is from about 2 to about 10 cycles. In some embodiments, the evaporation is accomplished with a rotary evaporator. In some embodiments, the evaporation is under reduced pressure.

### DETAILED DESCRIPTION OF THE DISCLOSURE

Interest in PROTACs as a new therapeutic modality has progressed rapidly over the past few years. Nonetheless, this new modality faces multiple challenges in drug delivery based on the poor physical properties of PROTACs as compared to traditional small molecule drugs. In general, PROTACs suffer from higher molecular weights, greater lipophilicity, and poor aqueous solubility; all of which can lead to issues with absorption, distribution, metabolism, and toxicity. Most PROTAC programs are working towards eventual oral delivery and, as a result, poor oral bioavailability becomes an issue leading to problems in understanding pharmcokinetics/pharmacodynamics (PK/PD) and translating pharmacology to higher species. An alternative delivery method would allow the use of novel delivery methods beyond the traditional oral formulations.

Incorporation of PROTACs into albumin nanoparticles as described herein, solves most of the problems for efficient delivery of these drugs, while retaining compound potency. Albumin nanoparticle formulations can incorporate compounds with high molecular weights, typically well in excess of 500 m.w., that are difficult or impossible to deliver as a traditional oral formulation. Similarly, typical PROTACs with high lipophilicity and poor aqueous solubility are well accommodated in an albumin nanoparticle, typically showing complete solubility in biocompatible aqueous solutions such as saline, 5% dextrose, or water. Thus, the albumin nanoparticle formulations described herein can overcome the issues of absorption, distribution, metabolism, and toxicity that the PROTAC class of compounds face, while retaining the physical properties that lead to mechanistic efficacy.

This application recognizes the use of nanoparticles as a drug delivery platform is an attractive approach as nanoparticles provide the following advantages: more specific drug targeting and delivery, reduction in toxicity while maintaining therapeutic effects, greater safety and biocompatibility, and faster development of new safe medicines. The use of a pharmaceutically acceptable carrier, such as a protein, is also advantageous as proteins, such as albumin, are nontoxic, non-immunogenic, biocompatible, and biodegradable.

Provided herein are compositions comprising nanoparticles that allow for the drug delivery of the compounds of Formula (I) described herein, which are heterobifunctional molecules comprising a compound that binds to a target protein, a linker, and a compound that binds to an E3 ubiquitin ligase. These nanoparticle compositions further comprise pharmaceutically acceptable carriers that interact with the compounds described herein to provide the compositions in a form that is suitable for administration to a subject in need thereof. In some embodiments, this application recognizes that the compounds of Formula (I) described herein, with specific pharmaceutically acceptable carriers, such as the albumin-based pharmaceutically acceptable carriers described herein, provide nanoparticle formulations that are stable.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range varies between 1% and 15% of the stated number or numerical range. The term “comprising” (and related terms such as “comprise” or “comprises” or “having” or “including”) is not intended to exclude that which in other certain embodiments, for example, an embodiment of any composition of matter, composition, method, or process, or the like, described herein, may “consist of” or “consist essentially of” the described features.

### Definitions

As used in the specification and appended claims, unless specified to the contrary, the following terms have the meaning indicated below.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist.

The term “target protein” as used herein, refers to a protein or polypeptide, which is a target for binding to a compound according to the present invention and degradation by ubiquitin ligase hereunder. Such small molecule target protein binding moieties (ligand B as defined in Formula (I) herein) also include pharmaceutically acceptable salts, enantiomers, solvates and polymorphs of these compositions, as well as other small molecules that may target a protein of interest. These binding moieties (B groups described in Formula (I) herein) are linked to a compound that binds to an E3 ubiquitin ligase (A groups described in Formula (I) herein) through a linker (L groups described in Formula (I) herein).

In some embodiments target proteins include, but are not limited to, structural proteins, receptors, enzymes, cell surface proteins, proteins pertinent to the integrated function of a cell, including proteins involved in catalytic activity, aromatase activity, motor activity, helicase activity, metabolic processes (anabolism and catrabolism), antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, translation regulator activity. Proteins of interest can include proteins from eurkaryotes and prokaryotes including humans as targets for drug therapy, other animals, including domesticated animals, microbials for the determination of targets for antibiotics and other antimicrobials and plants, and even viruses, among numerous others.

In some embodiments, target proteins include proteins which may be used to restore function in numerous polygenic diseases, including for example B7.1 and B7, TINFR1m, TNFR2, NADPH oxidase, BclIBax and other partners in the apotosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VCAM, VLA-4 integrin, selectins, CD40/CD40L, newokinins and receptors, inosine monophosphate dehydrogenase, p38 MAP Kinase, RaslRaflMEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors, adenosine receptors, adenosine kinase and AMP deaminase, purinergic receptors (P2Y1, P2Y2, P2Y4, P2Y6, P2X1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

“Optional” or “optionally” means that a subsequently described event or circumstance may or may not occur and that the description includes instances when the event or circumstance occurs and instances in which it does not. For example, “optionally substituted aryl” means that the aryl radical are or are not substituted and that the description includes both substituted aryl radicals and aryl radicals having no substitution.

As used herein, “treatment” or “treating” or “palliating” or “ameliorating” are used interchangeably herein. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has not been made.

### Compounds

The compounds of Formula (I) described herein are heterobifunctional molecules comprising a compound that binds to a target protein, a linker, and a compound that binds to an E3 ubiquitin ligase. As described herein, the compound of Formula (I) has the structure:

A-L-B Formula (I);

wherein:
A is a compound that binds to an E3 ubiquitin ligase;
L is a linker comprising at least two carbon atoms; and
B is a ligand which binds to a target protein or polypeptide which is to be mono-ubiquitinated or poly-ubiquitinated by the E3 ligase and thereby degraded, and is linked to the A group through the L group.

In some embodiments, A is selected from a cereblon binder, a Von Hippel-Lindau tumor suppressor protein (VHL) binder, an inhibitor of apoptosis protein (IAP) binder, a Kelch-like ECH-associated protein 1 (Keap1) binder, a mouse double minute 2 homolog (MDM2) binder, and beta-transducin repeat containing protein (b-TrCP) binder.

In some embodiments, A is a cereblon binder. In some embodiments, A is a cereblon binder selected from lenalidomide, pomalidomide, and thalidomide. In some embodiments, A is lenalidomide. In some embodiments, A is pomalidomide. In some embodiments, A is thalidomide.

In some embodiments, A is a VHL binder.

In some embodiments, A is an IAP binder. In some embodiments, A is an IAP binder selected from an X-linked inhibitor of apoptosis protein (XIAP), cellular inhibitor of apoptosis protein-1 (cIAP1), cellular inhibitor of apoptosis protein-2 (cIAP2), neuronal apoptosis inhibitory protein (NAIP), livin, and survivin. In some embodiments, A is an X-linked inhibitor of apoptosis protein (XIAP). In some embodiments, A is a cellular inhibitor of apoptosis protein-1 (cIAP1). In some embodiments, A is a cellular inhibitor of apoptosis protein-2 (cIAP2). In some embodiments, A is an IAP binder selected from a neuronal apoptosis inhibitory protein (NAIP). In some embodiments, A is livin. In some embodiments, A is survivin.

In some embodiments, A is a Keap1 binder.

In some embodiments, A is an MDM2 binder.

In some embodiments, A is a b-TrCP binder.

In some embodiments, L is a linker comprising at least two carbon atoms. In some embodiments, L is a linker comprising at least three carbon atoms. In some embodiments, L is a linker comprising at least four carbon atoms. In some embodiments, L is a linker comprising at least five carbon atoms. In some embodiments, L is a linker comprising at least six carbon atoms. In some embodiments, L is a linker comprising at least seven carbon atoms. In some embodiments, L is a linker comprising at least eight carbon atoms. In some embodiments, L is a linker comprising at least nine carbon atoms. In some embodiments, L is a linker comprising at least ten carbon atoms. In some embodiments, L is a linker comprising at least eleven carbon atoms. In some embodiments, L is a linker comprising at least twelve carbon atoms. In some embodiments, L is a linker comprising at least thirteen carbon atoms. In some embodiments, L is a linker comprising at least fourteen carbon atoms. In some embodiments, L is a linker comprising at least fifteen carbon atoms. In some embodiments, L is a linker comprising at least sixteen carbon atoms. In some embodiments, L is a linker comprising at least seventeen carbon atoms. In some embodiments, L is a linker comprising at least eighteen carbon atoms. In some embodiments, L is a linker comprising at least nineteen carbon atoms. In some embodiments, L is a linker comprising at least twenty carbon atoms.

In some embodiments, L is a linker comprising 2 to 20 carbon atoms. In some embodiments, L is a linker comprising 2 to 18 carbon atoms. In some embodiments, L is a linker comprising 2 to 16 carbon atoms. In some embodiments, L is a linker comprising 2 to 14 carbon atoms. In some embodiments, L is a linker comprising 2 to 12 carbon atoms. In some embodiments, L is a linker comprising 2 to 10 carbon atoms. In some embodiments, L is a linker comprising 2 to 9 carbon atoms. In some embodiments, L is a linker comprising 2 to 8 carbon atoms. In some embodiments, L is a linker comprising 2 to 7 carbon atoms. In some embodiments, L is a linker comprising 2 to 6 carbon atoms. In some embodiments, L is a linker comprising 2 to 5 carbon atoms. In some embodiments, L is a linker comprising 2 to 4 carbon atoms.

In some embodiments, L is a linker comprising 4 to 20 carbon atoms. In some embodiments, L is a linker comprising 4 to 18 carbon atoms. In some embodiments, L is a linker comprising 4 to 16 carbon atoms. In some embodiments, L is a linker comprising 4 to 14 carbon atoms. In some embodiments, L is a linker comprising 4 to 12 carbon atoms. In some embodiments, L is a linker comprising 4 to 10 carbon atoms. In some embodiments, L is a linker comprising 4 to 9 carbon atoms. In some embodiments, L is a linker comprising 4 to 8 carbon atoms. In some embodiments, L is a linker comprising 4 to 7 carbon atoms. In some embodiments, L is a linker comprising 4 to 6 carbon atoms.

In some embodiments, L is a linker comprising 6 to 20 carbon atoms. In some embodiments, L is a linker comprising 6 to 18 carbon atoms. In some embodiments, L is a linker comprising 6 to 16 carbon atoms. In some embodiments, L is a linker comprising 6 to 14 carbon atoms. In some embodiments, L is a linker comprising 6 to 12 carbon atoms. In some embodiments, L is a linker comprising 6 to 10 carbon atoms. In some embodiments, L is a linker comprising 6 to 9 carbon atoms. In some embodiments, L is a linker comprising 6 to 8 carbon atoms.

In some embodiments, L is a linker comprising at least two carbon atoms and at least one oxygen atom. In some embodiments, L is a linker comprising at least three carbon atoms and at least one oxygen atom. In some embodiments, L is a linker comprising at least four carbon atoms and at least one oxygen atom. In some embodiments, L is a linker comprising at least five carbon atoms and at least one oxygen atom. In some embodiments, L is a linker comprising at least six carbon atoms and at least one oxygen atom. In some embodiments, L is a linker comprising at least seven carbon atoms and at least one oxygen atom. In some embodiments, L is a linker comprising at least eight carbon atoms and at least one oxygen atom. In some embodiments, L is a linker comprising at least nine carbon atoms and at least one oxygen atom. In some embodiments, L is a linker comprising at least ten carbon atoms and at least one oxygen atom. In some embodiments, L is a linker comprising at least eleven carbon atoms and at least one oxygen atom. In some embodiments, L is a linker comprising at least twelve carbon atoms and at least one oxygen atom. In some embodiments, L is a linker comprising at least thirteen carbon atoms and at least one oxygen atom. In some embodiments, L is a linker comprising at least fourteen carbon atoms and at least one oxygen atom. In some embodiments, L is a linker comprising at least fifteen carbon atoms and at least one oxygen atom. In some embodiments, L is a linker comprising at least sixteen carbon atoms and at least one oxygen atom. In some embodiments, L is a linker comprising at least seventeen carbon atoms and at least one oxygen atom. In some embodiments, L is a linker comprising at least eighteen carbon atoms and at least one oxygen atom. In some embodiments, L is a linker comprising at least nineteen carbon atoms and at least one oxygen atom. In some embodiments, L is a linker comprising at least twenty carbon atoms and at least one oxygen atom.

In some embodiments, L is a linker comprising 2 to 20 carbon atoms and 1-8 oxygen atoms. In some embodiments, L is a linker comprising 2 to 18 carbon atoms and 1-6 oxygen atoms. In some embodiments, L is a linker comprising 2 to 16 carbon atoms and 1-6 oxygen atoms. In some embodiments, L is a linker comprising 2 to 14 carbon atoms and 1-6 oxygen atoms. In some embodiments, L is a linker comprising 2 to 12 carbon atoms and 1-6 oxygen atoms. In some embodiments, L is a linker comprising 2 to 10 carbon atoms and 1-5 oxygen atoms. In some embodiments, L is a linker comprising 2 to 9 carbon atoms and 1-4 oxygen atoms. In some embodiments, L is a linker comprising 2 to 8 carbon atoms and 1-4 oxygen atoms. In some embodiments, L is a linker comprising 2 to 7 carbon atoms and 1-4 oxygen atoms. In some embodiments, L is a linker comprising 2 to 6 carbon atoms and 1-4 oxygen atoms. In some embodiments, L is a linker comprising 2 to 5 carbon atoms and 1-3 oxygen atoms. In some embodiments, L is a linker comprising 2 to 4 carbon atoms and 1-3 oxygen atoms.

In some embodiments, L is a linker comprising 4 to 20 carbon atoms and 1-8 oxygen atoms. In some embodiments, L is a linker comprising 4 to 18 carbon atoms and 1-6 oxygen atoms. In some embodiments, L is a linker comprising 4 to 16 carbon atoms and 1-6 oxygen atoms. In some embodiments, L is a linker comprising 4 to 14 carbon atoms and 1-6 oxygen atoms. In some embodiments, L is a linker comprising 4 to 12 carbon atoms and 1-6 oxygen atoms. In some embodiments, L is a linker comprising 4 to 10 carbon atoms and 1-5 oxygen atoms. In some embodiments, L is a linker comprising 4 to 9 carbon atoms and 1-4 oxygen atoms. In some embodiments, L is a linker comprising 4 to 8 carbon atoms and 1-4 oxygen atoms. In some embodiments, L is a linker comprising 4 to 7 carbon atoms and 1-4 oxygen atoms. In some embodiments, L is a linker comprising 4 to 6 carbon atoms and 1-4 oxygen atoms.

In some embodiments of any of the linkers described herein, the linker is fully saturated. In some embodiments of any of the linkers described herein, the linker further comprises at least one alkenyl (carbon-carbon double bond) group. In some embodiments of any of the linkers described herein, the linker further comprises one alkenyl group. In some embodiments of any of the linkers described herein, the linker further comprises two alkenyl groups. In some embodiments of any of the linkers described herein, the linker further comprises at least one alkynyl (carbon-carbon triple bond) group. In some embodiments of any of the linkers described herein, the linker further comprises one alkynyl group. In some embodiments of any of the linkers described herein, the linker further comprises two alkynyl groups.

In some embodiments of any of the linkers described herein, the linker further comprises at least one —S— group. In some embodiments of any of the linkers described herein, the linker further comprises at least two —S— groups. In some embodiments of any of the linkers described herein, the linker further comprises at least three —S— groups. In some embodiments of any of the linkers described herein, the linker further comprises at least four —S— groups. In some embodiments of any of the linkers described herein, the linker further comprises one or two —S— groups. In some embodiments of any of the linkers described herein, the linker further comprises one —S— group. In some embodiments of any of the linkers described herein, the linker further comprises two —S— groups.

In some embodiments of any of the linkers described herein, the linker further comprises at least one —N(H)— group. In some embodiments of any of the linkers described herein, the linker further comprises at least two —N(H)— groups. In some embodiments of any of the linkers described herein, the linker further comprises at least three —N(H)— groups. In some embodiments of any of the linkers described herein, the linker further comprises at least four —N(H)— groups. In some embodiments of any of the linkers described herein, the linker further comprises one or two —N(H)— groups. In some embodiments of any of the linkers described herein, the linker further comprises one —N(H)— group. In some embodiments of any of the linkers described herein, the linker further comprises two —N(H)— groups.

In some embodiments of any of the linkers described herein, the linker further comprises at least one —C(O)N(H)— group. In some embodiments of any of the linkers described herein, the linker further comprises at least two —C(O)N(H)— groups. In some embodiments of any of the linkers described herein, the linker further comprises one or two —C(O)N(H)— groups. In some embodiments of any of the linkers described herein, the linker further comprises one —C(O)N(H)— group. In some embodiments of any of the linkers described herein, the linker further comprises two —C(O)N(H)— groups.

In some embodiments of any of the linkers described herein, the linker further comprises at least one —C(O)— group. In some embodiments of any of the linkers described herein, the linker further comprises at least two —C(O)— groups. In some embodiments of any of the linkers described herein, the linker further comprises one or two —C(O)— groups. In some embodiments of any of the linkers described herein, the linker further comprises one —C(O)— group. In some embodiments of any of the linkers described herein, the linker further comprises two —C(O)— groups.

In some embodiments of any of the linkers described herein, the linker further comprises at least one phenyl ring. In some embodiments of any of the linkers described herein, the linker further comprises one phenyl ring. In some embodiments of any of the linkers described herein, the linker further comprises two phenyl rings. In some embodiments of any of the linkers described herein, the linker further comprises at least one heteroaryl ring. In some embodiments of any of the linkers described herein, the linker further comprises one heteroaryl ring. In some embodiments of any of the linkers described herein, the linker further comprises two heteroaryl rings. In some embodiments of any of the linkers described herein, the linker further comprises a phenyl ring and a heteroaryl ring.

In some embodiments of any of the linkers described herein, the linker is unsubstituted. In some embodiments of any of the linkers described herein, the linker is substituted. In some embodiments of any of the linkers described herein, the linker is substituted with one or more groups selected from hydroxy, alkoxy, amino, alkylamino, di-alkylamino, alkyl, acyl, amido, carboxy, carboxylic ester, phenyl, cycloalkyl, heterocycloalkyl, and heteroaryl.

In some embodiments, the linker, L, is described in US20150291562, US20170281784, US20190142961, US20190144442, US20180228907, US20180215731, US20180125821, US20180099940, US20190210996, US20190152946, US20190119271, US20170121321, US20170065719, US20170037004, US20180147202, and US20180118733, each of which is incorporated by reference.

In some embodiments, B is a ligand which binds to a target protein or polypeptide which is to be mono-ubiquitinated or poly-ubiquitinated by the E3 ligase and thereby degraded, and is linked to the A group through the L group. In some embodiments, B is a ligand which binds to a target protein which is to be mono-ubiquitinated by the E3 ligase and thereby degraded, and is linked to the A group through the L group. In some embodiments, B is a ligand which binds to a target protein or polypeptide which is to be poly-ubiquitinated by the E3 ligase and thereby degraded, and is linked to the A group through the L group. In some embodiments, B is a ligand which binds to a target polypeptide which is to be mono-ubiquitinated by the E3 ligase and thereby degraded, and is linked to the A group through the L group. In some embodiments, B is a ligand which binds to a target polypeptide which is to be poly-ubiquitinated by the E3 ligase and thereby degraded, and is linked to the A group through the L group.

In some embodiments, ligand B reversibly binds to the the target target protein or polypeptide. In some embodiments, ligand B irreversibly binds to the the target target protein or polypeptide.

In some embodiments, B is selected from Hsp90 inhibitors, kinase inhibitors, MDM2 inhibitors, compounds targeting Human BET Bromodomain-containing proteins, HDAC inhibitors, human lysine methyltransferase inhibitors, angiogenesis inhibitors, immunosuppressive compounds, and compounds targeting the aryl hydrocarbon receptor (AHR).

In some embodiments, B is selected from an anti-cancer agent including, but not limited to, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitor, an AKT inhibitor, an mTORC1/2 inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 1314-TM-601, ALT-110, BIO 140, CC 8490, cilengitide, gimatecan, IL13-PE38QQR, INO 1001, IPdR.sub.1 KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]-benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

In some embodiments, ligand B is a compound targeting BET1. In some embodiments, ligand B is a compound targeting BRD4. In some embodiments, ligand B is a compound targeting CDK9.

In some embodiments, the ligand which binds to a target protein or polypeptide is described in US20150291562, US20170281784, US20190142961, US20190144442, US20180228907, US20180215731, US20180125821, US20180099940, US20190210996, US20190152946, US20190119271, US20170121321, US20170065719, US20170037004, US20180147202, and US20180118733, each of which is incorporated by reference.

In some embodiments, the compound of Formula (I) is:

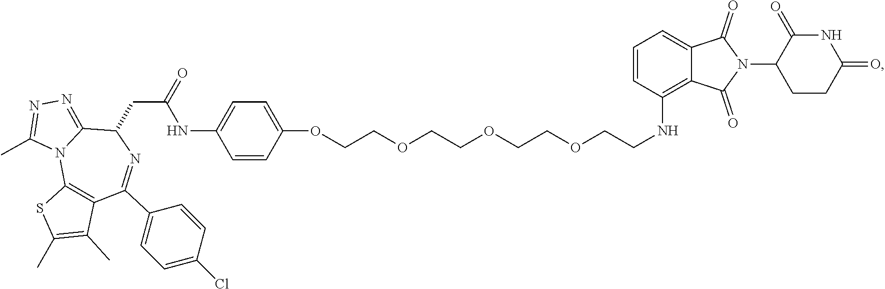 [SMILES: Cc1sc2c(c1C)C(c1ccc(Cl)cc1)=N[C@@H](CC(=O)Nc1ccc(OCCOCCOCCOCCNc3cccc4c3C(=O)N(C3CCC(=O)NC3=O)C4=O)cc1)c1nnc(C)n1-2]

or the pharmaceutically acceptable salt thereof.

In some embodiments, the compound of Formula (I) is:

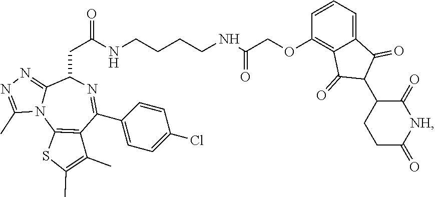 [SMILES: Cc1sc2c(c1C)C(c1ccc(Cl)cc1)=N[C@@H](CC(=O)NCCCCNC(=O)COc1cccc3c1C(=O)C(C1CCC(=O)NC1=O)C3=O)c1nnc(C)n1-2]

or the pharmaceutically acceptable salt thereof.

In some embodiments, the compound of Formula (I) is:

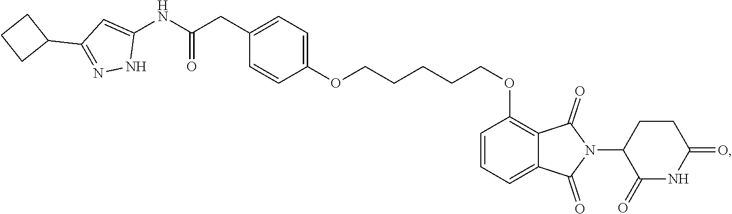 [SMILES: O=C1CCC(N2C(=O)c3cccc(OCCCCCOc4ccc(CC(=O)Nc5cc(C6CCC6)n[nH]5)cc4)c3C2=O)C(=O)N1]

or the pharmaceutically acceptable salt thereof.

In some embodiments, the compound of Formula (I) is:

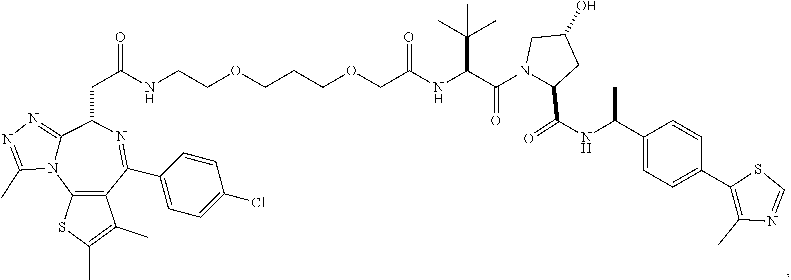 [SMILES: Cc1ncsc1-c1ccc([C@H](C)NC(=O)[C@@H]2C[C@@H](O)CN2C(=O)[C@@H](NC(=O)COCCCOCCNC(=O)C[C@@H]2N=C(c3ccc(Cl)cc3)c3c(sc(C)c3C)-n3c(C)nnc32)C(C)(C)C)cc1]

or the pharmaceutically acceptable salt thereof.

In some embodiments, the compound of Formula (I) is:

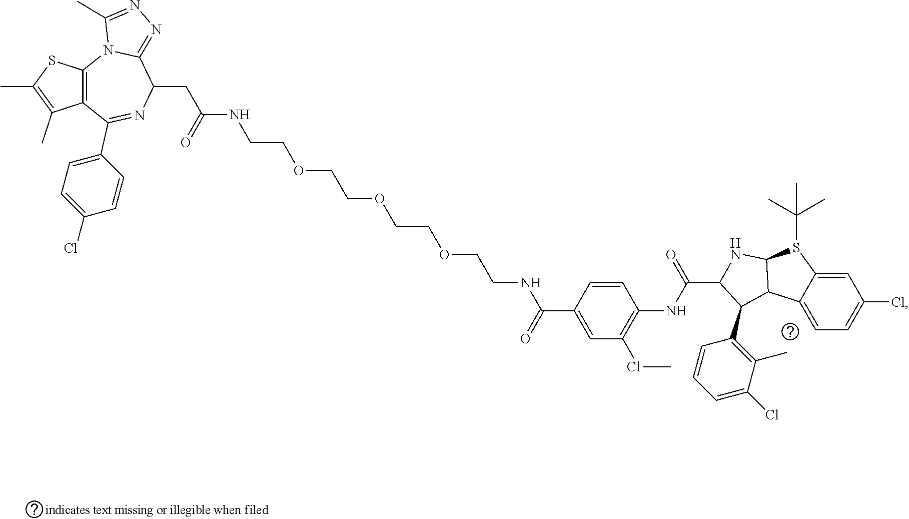

or the pharmaceutically acceptable salt thereof.

In some embodiments, the compound of Formula (I) is:

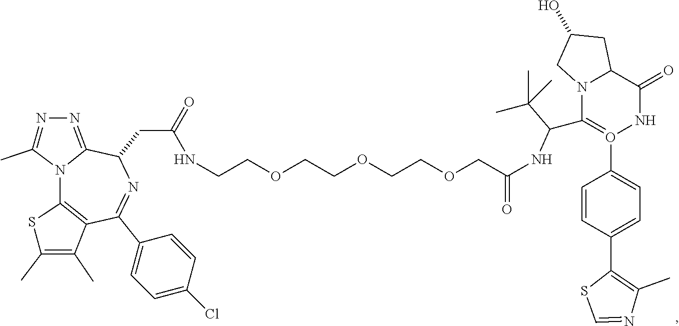

or the pharmaceutically acceptable salt thereof.

In some embodiments, the compound of Formula (I) is:

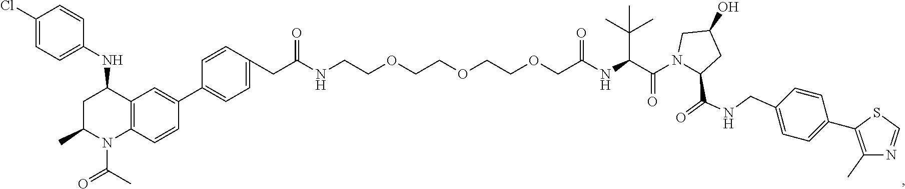 [SMILES: CC(=O)N1c2ccc(-c3ccc(CC(=O)NCCOCCOCCOCC(=O)N[C@H](C(=O)N4C[C@@H](O)C[C@H]4C(=O)NCc4ccc(-c5scnc5C)cc4)C(C)(C)C)cc3)cc2[C@H](Nc2ccc(Cl)cc2)C[C@@H]1C]

or the pharmaceutically acceptable salt thereof.

### Preparation of Compounds

The compounds used in the reactions described herein are made according to organic synthesis techniques, starting from commercially available chemicals and/or from compounds described in the chemical literature. “Commercially available chemicals” are obtained from standard commercial sources include, but are not limited to, Acros Organics (Geel, Belgium), Aldrich Chemical (Milwaukee, Wis., including Sigma Chemical and Fluka), Apin Chemicals Ltd. (Milton Park, UK), Ark Pharm, Inc. (Libertyville, Ill.), Avocado Research (Lancashire, U.K.), BDH Inc. (Toronto, Canada), Bionet (Cornwall, U.K.), Chemitek (Indianapolis, Ind.), Chemservice Inc. (West Chester, Pa.), Combi-blocks (San Diego, Calif.), Crescent Chemical Co. (Hauppauge, N.Y.), eMolecules (San Diego, Calif.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Matrix Scientific, (Columbia, S.C.), Maybridge Chemical Co. Ltd. (Cornwall, U.K.), MedChemExpress (Monmouth Junction, N.J.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Ryan Scientific, Inc. (Mount Pleasant, S.C.), Spectrum Chemicals (Gardena, Calif.), Sundia Meditech, (Shanghai, China), TCI America (Portland, Oreg.), Trans World Chemicals, Inc. (Rockville, Md.), and WuXi (Shanghai, China).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C.). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Further Forms of Compounds Disclosed Herein

### Isomers

In some embodiments, the compounds disclosed herein contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion, are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that does not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds described herein include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate, or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds described herein, or by separately reacting a purified compound in its free form with a suitable acid or base, and isolating the salt thus formed.

### Solvates

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or MeOH. In addition, the compounds provided herein exist in unsolvated as well as solvated forms. In general, the solvated forms are considered equivalent to the unsolvated forms for the purposes of the compounds and methods provided herein.

### Prodrugs

In some embodiments, compounds described herein are prepared as prodrugs. A “prodrug” refers to an agent that is converted into the parent drug in vivo. Prodrugs are often useful because, in some situations, they are easier to administer than the parent drug. In some embodiments, the prodrug is a substrate for a transporter. In some embodiments, the prodrug also has improved solubility in pharmaceutical compositions over the parent drug. In some embodiments, the design of a prodrug increases the effective water solubility. In some embodiments, the design of a prodrug decreases the effective water solubility. An example, without limitation, of a prodrug is a compound described herein, which is administered as an ester (the “prodrug”) but then is metabolically hydrolyzed to provide the active entity. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrug forms of the herein described compounds, wherein the prodrug is metabolized in vivo to produce a compound described herein as set forth herein are included within the scope of the claims. In some cases, some of the herein-described compounds is a prodrug for another derivative or active compound.

### Metabolites

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

### Pharmaceutically Acceptable Carrier

In some embodiments, the composition described herein also comprise a pharmaceutically acceptable carrier. In some embodiments, the pharmaceutically acceptable carrier is a protein. The term “protein’ as used herein refers to polypeptides or polymers comprising of amino acids of any length (including full length or fragments). These polypeptides or polymers are linear or branched, comprise modified amino acids, and/or are interrupted by non-amino acids. The term also encompasses an amino acid polymer that has been modified by natural means or by chemical modification. Examples of chemical modifications include, but are not limited to, disulfide bond formation, glycosylation, lipidation, acetylation, phosphorylation, or any other manipulation or modification. Also included within this term are, for example, polypeptides containing one or more analogs of an amino acid (including, for example, unnatural amino acids, etc.), as well as other modifications known in the art. The proteins described herein may be naturally occurring, i.e., obtained or derived from a natural source (such as blood), or synthesized (such as chemically synthesized or synthesized by recombinant DNA techniques). In some embodiments, the protein is naturally occurring. In some embodiments, the protein is obtained or derived from a natural source. In some embodiments, the protein is synthetically prepared.

Examples of suitable pharmaceutically acceptable carriers include proteins normally found in blood or plasma, such as albumin, immunoglobulin including IgA, lipoproteins, apolipoprotein B, alpha-acid glycoprotein, beta-2-macroglobulin, thyroglobulin, transferin, fibronectin, factor VII, factor VIII, factor IX, factor X, and the like. In some embodiments, the pharmaceutically acceptable carrier is a non-blood protein. Examples of non-blood protein include but are not limited to casein, C.-lactalbumin, and B-lactoglobulin.

In some embodiments, the pharmaceutically acceptable carrier is albumin. In some embodiments, the albumin is human serum albumin (HSA). Human serum albumin is the most abundant protein in human blood and is a highly soluble globular protein that consists of 585 amino acids and has a molecular weight of 66.5 kDa. Other albumins suitable for use include, but are not limited to, bovine serum albumin.

In some non-limiting embodiments, the composition described herein further comprises one or more albumin stabilizers. In some embodiments, the albumin stabilizer is N-acetyl tryptophan, octanoate salts, or a combination thereof.

In some embodiments, the molar ratio of the compound to pharmaceutically acceptable carrier is from about 1:1 to about 40:1. In some embodiments, the molar ratio of the compound to pharmaceutically acceptable carrier is from about 1:1 to about 20:1. In some embodiments, the molar ratio of the compound to pharmaceutically acceptable carrier is from about 2:1 to about 12:1.

In some embodiments, the molar ratio of the compound to pharmaceutically acceptable carrier is about 40:1. In some embodiments, the molar ratio of the compound to pharmaceutically acceptable carrier is about 35:1. In some embodiments, the molar ratio of the compound to pharmaceutically acceptable carrier is about 30:1. In some embodiments, the molar ratio of the compound to pharmaceutically acceptable carrier is about 25:1. In some embodiments, the molar ratio of the compound to pharmaceutically acceptable carrier is about 20:1. In some embodiments, the molar ratio of the compound to pharmaceutically acceptable carrier is about 19:1. In some embodiments, the molar ratio of the compound to pharmaceutically acceptable carrier is about 18:1. In some embodiments, the molar ratio of the compound to pharmaceutically acceptable carrier is about 17:1. In some embodiments, the molar ratio of the compound to pharmaceutically acceptable carrier is about 16:1. In some embodiments, the molar ratio of the compound to pharmaceutically acceptable carrier is about 15:1. In some embodiments, the molar ratio of the compound to pharmaceutically acceptable carrier is about 14:1. In some embodiments, the molar ratio of the compound to pharmaceutically acceptable carrier is about 13:1. In some embodiments, the molar ratio of the compound to pharmaceutically acceptable carrier is about 12:1. In some embodiments, the molar ratio of the compound to pharmaceutically acceptable carrier is about 11:1. In some embodiments, the molar ratio of the compound to pharmaceutically acceptable carrier is about 10:1. In some embodiments, the molar ratio of the compound to pharmaceutically acceptable carrier is about 9:1. In some embodiments, the molar ratio of the compound to pharmaceutically acceptable carrier is about 8:1. In some embodiments, the molar ratio of the compound to pharmaceutically acceptable carrier is about 7:1. In some embodiments, the molar ratio of the compound to pharmaceutically acceptable carrier is about 6:1. In some embodiments, the molar ratio of the compound to pharmaceutically acceptable carrier is about 5:1. In some embodiments, the molar ratio of the compound to pharmaceutically acceptable carrier is about 4:1. In some embodiments, the molar ratio of the compound to pharmaceutically acceptable carrier is about 3:1. In some embodiments, the molar ratio of the compound to pharmaceutically acceptable carrier is about 2:1.

### Nanoparticles

Described herein in one aspect is a composition comprising nanoparticles comprising a compound of Formula (I), or a pharmaceutically acceptable salt thereof, and a pharmaceutically acceptable carrier.

In some embodiments, the nanoparticles have an average diameter of about 1000 nm or less for a predetermined amount of time after nanoparticle formation. In some embodiments, the nanoparticles have an average diameter of about 950 nm or less for a predetermined amount of time after nanoparticle formation. In some embodiments, the nanoparticles have an average diameter of about 900 nm or less for a predetermined amount of time after nanoparticle formation. In some embodiments, the nanoparticles have an average diameter of about 850 nm or less for a predetermined amount of time after nanoparticle formation. In some embodiments, the nanoparticles have an average diameter of about 800 nm or less for a predetermined amount of time after nanoparticle formation. In some embodiments, the nanoparticles have an average diameter of about 750 nm or less for a predetermined amount of time after nanoparticle formation. In some embodiments, the nanoparticles have an average diameter of about 700 nm or less for a predetermined amount of time after nanoparticle formation. In some embodiments, the nanoparticles have an average diameter of about 650 nm or less for a predetermined amount of time after nanoparticle formation. In some embodiments, the nanoparticles have an average diameter of about 600 nm or less for a predetermined amount of time after nanoparticle formation. In some embodiments, the nanoparticles have an average diameter of about 550 nm or less for a predetermined amount of time after nanoparticle formation. In some embodiments, the nanoparticles have an average diameter of about 500 nm or less for a predetermined amount of time after nanoparticle formation. In some embodiments, the nanoparticles have an average diameter of about 450 nm or less for a predetermined amount of time after nanoparticle formation. In some embodiments, the nanoparticles have an average diameter of about 400 nm or less for a predetermined amount of time after nanoparticle formation. In some embodiments, the nanoparticles have an average diameter of about 350 nm or less for a predetermined amount of time after nanoparticle formation. In some embodiments, the nanoparticles have an average diameter of about 300 nm or less for a predetermined amount of time after nanoparticle formation. In some embodiments, the nanoparticles have an average diameter of about 250 nm or less for a predetermined amount of time after nanoparticle formation. In some embodiments, the nanoparticles have an average diameter of about 240 nm or less for a predetermined amount of time after nanoparticle formation. In some embodiments, the nanoparticles have an average diameter of about 230 nm or less for a predetermined amount of time after nanoparticle formation. In some embodiments, the nanoparticles have an average diameter of about 220 nm or less for a predetermined amount of time after nanoparticle formation. In some embodiments, the nanoparticles have an average diameter of about 210 nm or less for a predetermined amount of time after nanoparticle formation. In some embodiments, the nanoparticles have an average diameter of about 200 nm or less for a predetermined amount of time after nanoparticle formation. In some embodiments, the nanoparticles have an average diameter of about 190 nm or less for a predetermined amount of time after nanoparticle formation. In some embodiments, the nanoparticles have an average diameter of about 180 nm or less for a predetermined amount of time after nanoparticle formation. In some embodiments, the nanoparticles have an average diameter of about 170 nm or less for a predetermined amount of time after nanoparticle formation. In some embodiments, the nanoparticles have an average diameter of about 160 nm or less for a predetermined amount of time after nanoparticle formation. In some embodiments, the nanoparticles have an average diameter of about 150 nm or less for a predetermined amount of time after nanoparticle formation. In some embodiments, the nanoparticles have an average diameter of about 140 nm or less for a predetermined amount of time after nanoparticle formation. In some embodiments, the nanoparticles have an average diameter of about 130 nm or less for a predetermined amount of time after nanoparticle formation. In some embodiments, the nanoparticles have an average diameter of about 120 nm or less for a predetermined amount of time after nanoparticle formation. In some embodiments, the nanoparticles have an average diameter of about 110 nm or less for a predetermined amount of time after nanoparticle formation. In some embodiments, the nanoparticles have an average diameter of about 100 nm or less for a predetermined amount of time after nanoparticle formation. In some embodiments, the nanoparticles have an average diameter of about 90 nm or less for a predetermined amount of time after nanoparticle formation. In some embodiments, the nanoparticles have an average diameter of about 80 nm or less for a predetermined amount of time after nanoparticle formation. In some embodiments, the nanoparticles have an average diameter of about 70 nm or less for a predetermined amount of time after nanoparticle formation. In some embodiments, the nanoparticles have an average diameter of about 60 nm or less for a predetermined amount of time after nanoparticle formation. In some embodiments, the nanoparticles have an average diameter of about 50 nm or less for a predetermined amount of time after nanoparticle formation. In some embodiments, the nanoparticles have an average diameter of about 40 nm or less for a predetermined amount of time after nanoparticle formation. In some embodiments, the nanoparticles have an average diameter of about 30 nm or less for a predetermined amount of time after nanoparticle formation. In some embodiments, the nanoparticles have an average diameter of about 20 nm or less for a predetermined amount of time after nanoparticle formation. In some embodiments, the nanoparticles have an average diameter of about 10 nm or less for a predetermined amount of time after nanoparticle formation.

In some embodiments, the nanoparticles have an average diameter of about 10 nm or greater for a predetermined amount of time after nanoparticle formation. In some embodiments, the nanoparticles have an average diameter of about 20 nm or greater for a predetermined amount of time after nanoparticle formation. In some embodiments, the nanoparticles have an average diameter of about 30 nm or greater for a predetermined amount of time after nanoparticle formation. In some embodiments, the nanoparticles have an average diameter of about 40 nm or greater for a predetermined amount of time after nanoparticle formation. In some embodiments, the nanoparticles have an average diameter of about 50 nm or greater for a predetermined amount of time after nanoparticle formation. In some embodiments, the nanoparticles have an average diameter of about 60 nm or greater for a predetermined amount of time after nanoparticle formation. In some embodiments, the nanoparticles have an average diameter of about 70 nm or greater for a predetermined amount of time after nanoparticle formation. In some embodiments, the nanoparticles have an average diameter of about 80 nm or greater for a predetermined amount of time after nanoparticle formation. In some embodiments, the nanoparticles have an average diameter of about 90 nm or greater for a predetermined amount of time after nanoparticle formation. In some embodiments, the nanoparticles have an average diameter of about 100 nm or greater for a predetermined amount of time after nanoparticle formation. In some embodiments, the nanoparticles have an average diameter of about 110 nm or greater for a predetermined amount of time after nanoparticle formation. In some embodiments, the nanoparticles have an average diameter of about 120 nm or greater for a predetermined amount of time after nanoparticle formation. In some embodiments, the nanoparticles have an average diameter of about 130 nm or greater for a predetermined amount of time after nanoparticle formation. In some embodiments, the nanoparticles have an average diameter of about 140 nm or greater for a predetermined amount of time after nanoparticle formation. In some embodiments, the nanoparticles have an average diameter of about 150 nm or greater for a predetermined amount of time after nanoparticle formation. In some embodiments, the nanoparticles have an average diameter of about 160 nm or greater for a predetermined amount of time after nanoparticle formation. In some embodiments, the nanoparticles have an average diameter of about 170 nm or greater for a predetermined amount of time after nanoparticle formation. In some embodiments, the nanoparticles have an average diameter of about 180 nm or greater for a predetermined amount of time after nanoparticle formation. In some embodiments, the nanoparticles have an average diameter of about 190 nm or greater for a predetermined amount of time after nanoparticle formation. In some embodiments, the nanoparticles have an average diameter of about 200 nm or greater for a predetermined amount of time after nanoparticle formation. In some embodiments, the nanoparticles have an average diameter of about 210 nm or greater for a predetermined amount of time after nanoparticle formation. In some embodiments, the nanoparticles have an average diameter of about 220 nm or greater for a predetermined amount of time after nanoparticle formation. In some embodiments, the nanoparticles have an average diameter of about 230 nm or greater for a predetermined amount of time after nanoparticle formation. In some embodiments, the nanoparticles have an average diameter of about 240 nm or greater for a predetermined amount of time after nanoparticle formation. In some embodiments, the nanoparticles have an average diameter of about 250 nm or greater for a predetermined amount of time after nanoparticle formation. In some embodiments, the nanoparticles have an average diameter of about 300 nm or greater for a predetermined amount of time after nanoparticle formation. In some embodiments, the nanoparticles have an average diameter of about 350 nm or greater for a predetermined amount of time after nanoparticle formation. In some embodiments, the nanoparticles have an average diameter of about 400 nm or greater for a predetermined amount of time after nanoparticle formation. In some embodiments, the nanoparticles have an average diameter of about 450 nm or greater for a predetermined amount of time after nanoparticle formation. In some embodiments, the nanoparticles have an average diameter of about 500 nm or greater for a predetermined amount of time after nanoparticle formation. In some embodiments, the nanoparticles have an average diameter of about 550 nm or greater for a predetermined amount of time after nanoparticle formation. In some embodiments, the nanoparticles have an average diameter of about 600 nm or greater for a predetermined amount of time after nanoparticle formation. In some embodiments, the nanoparticles have an average diameter of about 650 nm or greater for a predetermined amount of time after nanoparticle formation. In some embodiments, the nanoparticles have an average diameter of about 700 nm or greater for a predetermined amount of time after nanoparticle formation. In some embodiments, the nanoparticles have an average diameter of about 750 nm or greater for a predetermined amount of time after nanoparticle formation. In some embodiments, the nanoparticles have an average diameter of about 800 nm or greater for a predetermined amount of time after nanoparticle formation. In some embodiments, the nanoparticles have an average diameter of about 850 nm or greater for a predetermined amount of time after nanoparticle formation. In some embodiments, the nanoparticles have an average diameter of about 900 nm or greater for a predetermined amount of time after nanoparticle formation. In some embodiments, the nanoparticles have an average diameter of about 950 nm or greater for a predetermined amount of time after nanoparticle formation

In some embodiments, the nanoparticles have an average diameter of from about 10 nm to about 1000 nm for a predetermined amount of time after nanoparticle formation. In some embodiments, the nanoparticles have an average diameter of from about 10 nm to about 950 nm for a predetermined amount of time after nanoparticle formation. In some embodiments, the nanoparticles have an average diameter of from about 10 nm to about 900 nm for a predetermined amount of time after nanoparticle formation. In some embodiments, the nanoparticles have an average diameter of from about 10 nm to about 850 nm for a predetermined amount of time after nanoparticle formation. In some embodiments, the nanoparticles have an average diameter of from about 10 nm to about 800 nm for a predetermined amount of time after nanoparticle formation. In some embodiments, the nanoparticles have an average diameter of from about 10 nm to about 750 nm for a predetermined amount of time after nanoparticle formation. In some embodiments, the nanoparticles have an average diameter of from about 10 nm to about 700 nm for a predetermined amount of time after nanoparticle formation. In some embodiments, the nanoparticles have an average diameter of from about 10 nm to about 650 nm for a predetermined amount of time after nanoparticle formation. In some embodiments, the nanoparticles have an average diameter of from about 10 nm to about 600 nm for a predetermined amount of time after nanoparticle formation. In some embodiments, the nanoparticles have an average diameter of from about 10 nm to about 550 nm for a predetermined amount of time after nanoparticle formation for a predetermined amount of time after nanoparticle formation. In some embodiments, the nanoparticles have an average diameter of from about 10 nm to about 500 nm for a predetermined amount of time after nanoparticle formation. In some embodiments, the nanoparticles have an average diameter of from about 10 nm to about 450 nm for a predetermined amount of time after nanoparticle formation. In some embodiments, the nanoparticles have an average diameter of from about 10 nm to about 400 nm for a predetermined amount of time after nanoparticle formation. In some embodiments, the nanoparticles have an average diameter of from about 10 nm to about 350 nm for a predetermined amount of time after nanoparticle formation. In some embodiments, the nanoparticles have an average diameter of from about 10 nm to about 300 nm for a predetermined amount of time after nanoparticle formation. In some embodiments, the nanoparticles have an average diameter of from about 10 nm to about 250 nm for a predetermined amount of time after nanoparticle formation. In some embodiments, the nanoparticles have an average diameter of from about 10 nm to about 240 nm for a predetermined amount of time after nanoparticle formation. In some embodiments, the nanoparticles have an average diameter of from about 10 nm to about 230 nm for a predetermined amount of time after nanoparticle formation. In some embodiments, the nanoparticles have an average diameter of from about 10 nm to about 220 nm for a predetermined amount of time after nanoparticle formation. In some embodiments, the nanoparticles have an average diameter of from about 10 nm to about 210 nm for a predetermined amount of time after nanoparticle formation. In some embodiments, the nanoparticles have an average diameter of from about 10 nm to about 200 nm for a predetermined amount of time after nanoparticle formation. In some embodiments, the nanoparticles have an average diameter of from about 10 nm to about 190 nm for a predetermined amount of time after nanoparticle formation. In some embodiments, the nanoparticles have an average diameter of from about 10 nm to about 180 nm for a predetermined amount of time after nanoparticle formation. In some embodiments, the nanoparticles have an average diameter of from about 10 nm to about 170 nm for a predetermined amount of time after nanoparticle formation. In some embodiments, the nanoparticles have an average diameter of from about 10 nm to about 160 nm for a predetermined amount of time after nanoparticle formation. In some embodiments, the nanoparticles have an average diameter of from about 10 nm to about 150 nm for a predetermined amount of time after nanoparticle formation. In some embodiments, the nanoparticles have an average diameter of from about 10 nm to about 140 nm for a predetermined amount of time after nanoparticle formation. In some embodiments, the nanoparticles have an average diameter of from about 10 nm to about 130 nm for a predetermined amount of time after nanoparticle formation. In some embodiments, the nanoparticles have an average diameter of from about 10 nm to about 120 nm for a predetermined amount of time after nanoparticle formation. In some embodiments, the nanoparticles have an average diameter of from about 10 nm to about 110 nm for a predetermined amount of time after nanoparticle formation. In some embodiments, the nanoparticles have an average diameter of from about 10 nm to about 100 nm for a predetermined amount of time after nanoparticle formation. In some embodiments, the nanoparticles have an average diameter of from about 10 nm to about 90 nm for a predetermined amount of time after nanoparticle formation. In some embodiments, the nanoparticles have an average diameter of from about 10 nm to about 80 nm for a predetermined amount of time after nanoparticle formation. In some embodiments, the nanoparticles have an average diameter of from about 10 nm to about 70 nm for a predetermined amount of time after nanoparticle formation. In some embodiments, the nanoparticles have an average diameter of from about 10 nm to about 60 nm for a predetermined amount of time after nanoparticle formation. In some embodiments, the nanoparticles have an average diameter of from about 10 nm to about 50 nm for a predetermined amount of time after nanoparticle formation. In some embodiments, the nanoparticles have an average diameter of from about 10 nm to about 40 nm for a predetermined amount of time after nanoparticle formation. In some embodiments, the nanoparticles have an average diameter of from about 10 nm to about 30 nm for a predetermined amount of time after nanoparticle formation. In some embodiments, the nanoparticles have an average diameter of from about 10 nm to about 20 nm for a predetermined amount of time after nanoparticle formation.

In some embodiments, the nanoparticles have an average diameter of about 10 nm for a predetermined amount of time after nanoparticle formation. In some embodiments, the nanoparticles have an average diameter of about 20 nm for a predetermined amount of time after nanoparticle formation. In some embodiments, the nanoparticles have an average diameter of about 30 nm for a predetermined amount of time after nanoparticle formation. In some embodiments, the nanoparticles have an average diameter of about 40 nm for a predetermined amount of time after nanoparticle formation. In some embodiments, the nanoparticles have an average diameter of about 50 nm for a predetermined amount of time after nanoparticle formation. In some embodiments, the nanoparticles have an average diameter of about 60 nm for a predetermined amount of time after nanoparticle formation. In some embodiments, the nanoparticles have an average diameter of about 70 nm for a predetermined amount of time after nanoparticle formation. In some embodiments, the nanoparticles have an average diameter of about 80 nm for a predetermined amount of time after nanoparticle formation. In some embodiments, the nanoparticles have an average diameter of about 90 nm for a predetermined amount of time after nanoparticle formation. In some embodiments, the nanoparticles have an average diameter of about 100 nm for a predetermined amount of time after nanoparticle formation. In some embodiments, the nanoparticles have an average diameter of about 110 nm for a predetermined amount of time after nanoparticle formation. In some embodiments, the nanoparticles have an average diameter of about 120 nm for a predetermined amount of time after nanoparticle formation. In some embodiments, the nanoparticles have an average diameter of about 130 nm for a predetermined amount of time after nanoparticle formation. In some embodiments, the nanoparticles have an average diameter of about 140 nm for a predetermined amount of time after nanoparticle formation. In some embodiments, the nanoparticles have an average diameter of about 150 nm for a predetermined amount of time after nanoparticle formation. In some embodiments, the nanoparticles have an average diameter of about 160 nm for a predetermined amount of time after nanoparticle formation. In some embodiments, the nanoparticles have an average diameter of about 170 nm for a predetermined amount of time after nanoparticle formation. In some embodiments, the nanoparticles have an average diameter of about 180 nm for a predetermined amount of time after nanoparticle formation. In some embodiments, the nanoparticles have an average diameter of about 190 nm for a predetermined amount of time after nanoparticle formation. In some embodiments, the nanoparticles have an average diameter of about 200 nm for a predetermined amount of time after nanoparticle formation. In some embodiments, the nanoparticles have an average diameter of about 210 nm for a predetermined amount of time after nanoparticle formation. In some embodiments, the nanoparticles have an average diameter of about 220 nm for a predetermined amount of time after nanoparticle formation. In some embodiments, the nanoparticles have an average diameter of about 230 nm for a predetermined amount of time after nanoparticle formation. In some embodiments, the nanoparticles have an average diameter of about 240 nm for a predetermined amount of time after nanoparticle formation. In some embodiments, the nanoparticles have an average diameter of about 250 nm for a predetermined amount of time after nanoparticle formation. In some embodiments, the nanoparticles have an average diameter of about 300 nm for a predetermined amount of time after nanoparticle formation. In some embodiments, the nanoparticles have an average diameter of about 350 nm for a predetermined amount of time after nanoparticle formation. In some embodiments, the nanoparticles have an average diameter of about 400 nm for a predetermined amount of time after nanoparticle formation. In some embodiments, the nanoparticles have an average diameter of about 450 nm for a predetermined amount of time after nanoparticle formation. In some embodiments, the nanoparticles have an average diameter of about 500 nm for a predetermined amount of time after nanoparticle formation. In some embodiments, the nanoparticles have an average diameter of about 550 nm for a predetermined amount of time after nanoparticle formation. In some embodiments, the nanoparticles have an average diameter of about 600 nm for a predetermined amount of time after nanoparticle formation. In some embodiments, the nanoparticles have an average diameter of about 650 nm for a predetermined amount of time after nanoparticle formation. In some embodiments, the nanoparticles have an average diameter of about 700 nm for a predetermined amount of time after nanoparticle formation. In some embodiments, the nanoparticles have an average diameter of about 750 nm for a predetermined amount of time after nanoparticle formation. In some embodiments, the nanoparticles have an average diameter of about 800 nm for a predetermined amount of time after nanoparticle formation. In some embodiments, the nanoparticles have an average diameter of about 850 nm for a predetermined amount of time after nanoparticle formation. In some embodiments, the nanoparticles have an average diameter of about 900 nm for a predetermined amount of time after nanoparticle formation. In some embodiments, the nanoparticles have an average diameter of about 950 nm for a predetermined amount of time after nanoparticle formation. In some embodiments, the nanoparticles have an average diameter of about 1000 nm for a predetermined amount of time after nanoparticle formation.

In some embodiments, the predetermined amount of time is at least about 15 minutes. In some embodiments, the predetermined amount of time is at least about 30 minutes. In some embodiments, the predetermined amount of time is at least about 45 minutes. In some embodiments, the predetermined amount of time is at least about 1 hour. In some embodiments, the predetermined amount of time is at least about 2 hours. In some embodiments, the predetermined amount of time is at least about 3 hours. In some embodiments, the predetermined amount of time is at least about 4 hours. In some embodiments, the predetermined amount of time is at least about 5 hours. In some embodiments, the predetermined amount of time is at least about 6 hours. In some embodiments, the predetermined amount of time is at least about 7 hours. In some embodiments, the predetermined amount of time is at least about 8 hours. In some embodiments, the predetermined amount of time is at least about 9 hours. In some embodiments, the predetermined amount of time is at least about 10 hours. In some embodiments, the predetermined amount of time is at least about 11 hours. In some embodiments, the predetermined amount of time is at least about 12 hours. In some embodiments, the predetermined amount of time is at least about 1 day. In some embodiments, the predetermined amount of time is at least about 2 days. In some embodiments, the predetermined amount of time is at least about 3 days. In some embodiments, the predetermined amount of time is at least about 4 days. In some embodiments, the predetermined amount of time is at least about 5 days. In some embodiments, the predetermined amount of time is at least about 6 days. In some embodiments, the predetermined amount of time is at least about 7 days. In some embodiments, the predetermined amount of time is at least about 14 days. In some embodiments, the predetermined amount of time is at least about 21 days. In some embodiments, the predetermined amount of time is at least about 30 days.

In some embodiments, the predetermined amount of time is from about 15 minutes to about 30 days. In some embodiments, the predetermined amount of time is about 30 minutes to about 30 days. In some embodiments, the predetermined amount of time is from about 45 minutes to about 30 days. In some embodiments, the predetermined amount of time is from about 1 hour to about 30 days. In some embodiments, the predetermined amount of time is from about 2 hours to about 30 days. In some embodiments, the predetermined amount of time is from about 3 hours to about 30 days. In some embodiments, the predetermined amount of time is from about 4 hours to about 30 days. In some embodiments, the predetermined amount of time is from about 5 hours to about 30 days. In some embodiments, the predetermined amount of time is from about 6 hours to about 30 days. In some embodiments, the predetermined amount of time is from about 7 hours to about 30 days. In some embodiments, the predetermined amount of time is from about 8 hours to about 30 days. In some embodiments, the predetermined amount of time is from about 9 hours to about 30 days. In some embodiments, the predetermined amount of time is from about 10 hours to about 30 days. In some embodiments, the predetermined amount of time is from about 11 hours to about 30 days. In some embodiments, the predetermined amount of time is from about 12 hours to about 30 days. In some embodiments, the predetermined amount of time is from about 1 day to about 30 days. In some embodiments, the predetermined amount of time is from about 2 days to about 30 days. In some embodiments, the predetermined amount of time is from about 3 days to about 30 days. In some embodiments, the predetermined amount of time is from about 4 days to about 30 days. In some embodiments, the predetermined amount of time is from about 5 days to about 30 days. In some embodiments, the predetermined amount of time is from about 6 days to about 30 days. In some embodiments, the predetermined amount of time is from about 7 days to about 30 days. In some embodiments, the predetermined amount of time is from about 14 days to about 30 days. In some embodiments, the predetermined amount of time is from about 21 days to about 30 days.

In some embodiments, the nanoparticles have an average diameter of about 1000 nm or less for at least about 15 minutes after nanoparticle formation. In some embodiments, the nanoparticles have an average diameter of about 950 nm or less for at least about 15 minutes after nanoparticle formation. In some embodiments, the nanoparticles have an average diameter of about 900 nm or less for at least about 15 minutes after nanoparticle formation. In some embodiments, the nanoparticles have an average diameter of about 850 nm or less for at least about 15 minutes after nanoparticle formation. In some embodiments, the nanoparticles have an average diameter of about 800 nm or less for at least about 15 minutes after nanoparticle formation. In some embodiments, the nanoparticles have an average diameter of about 750 nm or less for at least about 15 minutes after nanoparticle formation. In some embodiments, the nanoparticles have an average diameter of about 700 nm or less for at least about 15 minutes after nanoparticle formation. In some embodiments, the nanoparticles have an average diameter of about 650 nm or less for at least about 15 minutes after nanoparticle formation. In some embodiments, the nanoparticles have an average diameter of about 600 nm or less for at least about 15 minutes after nanoparticle formation. In some embodiments, the nanoparticles have an average diameter of about 550 nm or less for at least about 15 minutes after nanoparticle formation. In some embodiments, the nanoparticles have an average diameter of about 500 nm or less for at least about 15 minutes after nanoparticle formation. In some embodiments, the nanoparticles have an average diameter of about 450 nm or less for at least about 15 minutes after nanoparticle formation. In some embodiments, the nanoparticles have an average diameter of about 400 nm or less for at least about 15 minutes after nanoparticle formation. In some embodiments, the nanoparticles have an average diameter of about 350 nm or less for at least about 15 minutes after nanoparticle formation. In some embodiments, the nanoparticles have an average diameter of about 300 nm or less for at least about 15 minutes after nanoparticle formation. In some embodiments, the nanoparticles have an average diameter of about 250 nm or less for at least about 15 minutes after nanoparticle formation. In some embodiments, the nanoparticles have an average diameter of about 240 nm or less for at least about 15 minutes after nanoparticle formation. In some embodiments, the nanoparticles have an average diameter of about 230 nm or less for at least about 15 minutes after nanoparticle formation. In some embodiments, the nanoparticles have an average diameter of about 220 nm or less for at least about 15 minutes after nanoparticle formation. In some embodiments, the nanoparticles have an average diameter of about 210 nm or less for at least about 15 minutes after nanoparticle formation. In some embodiments, the nanoparticles have an average diameter of about 200 nm or less for at least about 15 minutes after nanoparticle formation. In some embodiments, the nanoparticles have an average diameter of about 190 nm or less for at least about 15 minutes after nanoparticle formation. In some embodiments, the nanoparticles have an average diameter of about 180 nm or less for at least about 15 minutes after nanoparticle formation. In some embodiments, the nanoparticles have an average diameter of about 170 nm or less for at least about 15 minutes after nanoparticle formation. In some embodiments, the nanoparticles have an average diameter of about 160 nm or less for at least about 15 minutes after nanoparticle formation. In some embodiments, the nanoparticles have an average diameter of about 150 nm or less for at least about 15 minutes after nanoparticle formation. In some embodiments, the nanoparticles have an average diameter of about 140 nm or less for at least about 15 minutes after nanoparticle formation. In some embodiments, the nanoparticles have an average diameter of about 130 nm or less for at least about 15 minutes after nanoparticle formation. In some embodiments, the nanoparticles have an average diameter of about 120 nm or less for at least about 15 minutes after nanoparticle formation. In some embodiments, the nanoparticles have an average diameter of about 110 nm or less for at least about 15 minutes after nanoparticle formation. In some embodiments, the nanoparticles have an average diameter of about 100 nm or less for at least about 15 minutes after nanoparticle formation. In some embodiments, the nanoparticles have an average diameter of about 90 nm or less for at least about 15 minutes after nanoparticle formation. In some embodiments, the nanoparticles have an average diameter of about 80 nm or less for at least about 15 minutes after nanoparticle formation. In some embodiments, the nanoparticles have an average diameter of about 70 nm or less for at least about 15 minutes after nanoparticle formation. In some embodiments, the nanoparticles have an average diameter of about 60 nm or less for at least about 15 minutes after nanoparticle formation. In some embodiments, the nanoparticles have an average diameter of about 50 nm or less for at least about 15 minutes after nanoparticle formation. In some embodiments, the nanoparticles have an average diameter of about 40 nm or less for at least about 15 minutes after nanoparticle formation. In some embodiments, the nanoparticles have an average diameter of about 30 nm or less for at least about 15 minutes after nanoparticle formation. In some embodiments, the nanoparticles have an average diameter of about 20 nm or less for at least about 15 minutes after nanoparticle formation. In some embodiments, the nanoparticles have an average diameter of about 10 nm or less for at least about 15 minutes after nanoparticle formation.

In some embodiments, the nanoparticles have an average diameter of about 10 nm or greater for at least about 15 minutes after nanoparticle formation. In some embodiments, the nanoparticles have an average diameter of about 20 nm or greater for at least about 15 minutes after nanoparticle formation. In some embodiments, the nanoparticles have an average diameter of about 30 nm or greater for at least about 15 minutes after nanoparticle formation. In some embodiments, the nanoparticles have an average diameter of about 40 nm or greater for at least about 15 minutes after nanoparticle formation. In some embodiments, the nanoparticles have an average diameter of about 50 nm or greater for at least about 15 minutes after nanoparticle formation. In some embodiments, the nanoparticles have an average diameter of about 60 nm or greater for at least about 15 minutes after nanoparticle formation. In some embodiments, the nanoparticles have an average diameter of about 70 nm or greater for at least about 15 minutes after nanoparticle formation. In some embodiments, the nanoparticles have an average diameter of about 80 nm or greater for at least about 15 minutes after nanoparticle formation. In some embodiments, the nanoparticles have an average diameter of about 90 nm or greater for at least about 15 minutes after nanoparticle formation. In some embodiments, the nanoparticles have an average diameter of about 100 nm or greater for at least about 15 minutes after nanoparticle formation. In some embodiments, the nanoparticles have an average diameter of about 110 nm or greater for at least about 15 minutes after nanoparticle formation. In some embodiments, the nanoparticles have an average diameter of about 120 nm or greater for at least about 15 minutes after nanoparticle formation. In some embodiments, the nanoparticles have an average diameter of about 130 nm or greater for at least about 15 minutes after nanoparticle formation. In some embodiments, the nanoparticles have an average diameter of about 140 nm or greater for at least about 15 minutes after nanoparticle formation. In some embodiments, the nanoparticles have an average diameter of about 150 nm or greater for at least about 15 minutes after nanoparticle formation. In some embodiments, the nanoparticles have an average diameter of about 160 nm or greater for at least about 15 minutes after nanoparticle formation. In some embodiments, the nanoparticles have an average diameter of about 170 nm or greater for at least about 15 minutes after nanoparticle formation. In some embodiments, the nanoparticles have an average diameter of about 180 nm or greater for at least about 15 minutes after nanoparticle formation. In some embodiments, the nanoparticles have an average diameter of about 190 nm or greater for at least about 15 minutes after nanoparticle formation. In some embodiments, the nanoparticles have an average diameter of about 200 nm or greater for at least about 15 minutes after nanoparticle formation. In some embodiments, the nanoparticles have an average diameter of about 210 nm or greater for at least about 15 minutes after nanoparticle formation. In some embodiments, the nanoparticles have an average diameter of about 220 nm or greater for at least about 15 minutes after nanoparticle formation. In some embodiments, the nanoparticles have an average diameter of about 230 nm or greater for at least about 15 minutes after nanoparticle formation. In some embodiments, the nanoparticles have an average diameter of about 240 nm or greater for at least about 15 minutes after nanoparticle formation. In some embodiments, the nanoparticles have an average diameter of about 250 nm or greater for at least about 15 minutes after nanoparticle formation. In some embodiments, the nanoparticles have an average diameter of about 300 nm or greater for at least about 15 minutes after nanoparticle formation. In some embodiments, the nanoparticles have an average diameter of about 350 nm or greater for at least about 15 minutes after nanoparticle formation. In some embodiments, the nanoparticles have an average diameter of about 400 nm or greater for at least about 15 minutes after nanoparticle formation. In some embodiments, the nanoparticles have an average diameter of about 450 nm or greater for at least about 15 minutes after nanoparticle formation. In some embodiments, the nanoparticles have an average diameter of about 500 nm or greater for at least about 15 minutes after nanoparticle formation. In some embodiments, the nanoparticles have an average diameter of about 550 nm or greater for at least about 15 minutes after nanoparticle formation. In some embodiments, the nanoparticles have an average diameter of about 600 nm or greater for at least about 15 minutes after nanoparticle formation. In some embodiments, the nanoparticles have an average diameter of about 650 nm or greater for at least about 15 minutes after nanoparticle formation. In some embodiments, the nanoparticles have an average diameter of about 700 nm or greater for at least about 15 minutes after nanoparticle formation. In some embodiments, the nanoparticles have an average diameter of about 750 nm or greater for at least about 15 minutes after nanoparticle formation. In some embodiments, the nanoparticles have an average diameter of about 800 nm or greater for at least about 15 minutes after nanoparticle formation. In some embodiments, the nanoparticles have an average diameter of about 850 nm or greater for at least about 15 minutes after nanoparticle formation. In some embodiments, the nanoparticles have an average diameter of about 900 nm or greater for at least about 15 minutes after nanoparticle formation. In some embodiments, the nanoparticles have an average diameter of about 950 nm or greater for at least about 15 minutes after nanoparticle formation

In some embodiments, the nanoparticles have an average diameter of from about 10 nm to about 1000 nm for at least about 15 minutes after nanoparticle formation. In some embodiments, the nanoparticles have an average diameter of from about 10 nm to about 950 nm for at least about 15 minutes after nanoparticle formation. In some embodiments, the nanoparticles have an average diameter of from about 10 nm to about 900 nm for at least about 15 minutes after nanoparticle formation. In some embodiments, the nanoparticles have an average diameter of from about 10 nm to about 850 nm for at least about 15 minutes after nanoparticle formation. In some embodiments, the nanoparticles have an average diameter of from about 10 nm to about 800 nm for at least about 15 minutes after nanoparticle formation. In some embodiments, the nanoparticles have an average diameter of from about 10 nm to about 750 nm for at least about 15 minutes after nanoparticle formation. In some embodiments, the nanoparticles have an average diameter of from about 10 nm to about 700 nm for at least about 15 minutes after nanoparticle formation. In some embodiments, the nanoparticles have an average diameter of from about 10 nm to about 650 nm for at least about 15 minutes after nanoparticle formation. In some embodiments, the nanoparticles have an average diameter of from about 10 nm to about 600 nm for at least about 15 minutes after nanoparticle formation. In some embodiments, the nanoparticles have an average diameter of from about 10 nm to about 550 nm for at least about 15 minutes after nanoparticle formation for at least about 15 minutes after nanoparticle formation. In some embodiments, the nanoparticles have an average diameter of from about 10 nm to about 500 nm for at least about 15 minutes after nanoparticle formation. In some embodiments, the nanoparticles have an average diameter of from about 10 nm to about 450 nm for at least about 15 minutes after nanoparticle formation. In some embodiments, the nanoparticles have an average diameter of from about 10 nm to about 400 nm for at least about 15 minutes after nanoparticle formation. In some embodiments, the nanoparticles have an average diameter of from about 10 nm to about 350 nm for at least about 15 minutes after nanoparticle formation. In some embodiments, the nanoparticles have an average diameter of from about 10 nm to about 300 nm for at least about 15 minutes after nanoparticle formation. In some embodiments, the nanoparticles have an average diameter of from about 10 nm to about 250 nm for at least about 15 minutes after nanoparticle formation. In some embodiments, the nanoparticles have an average diameter of from about 10 nm to about 240 nm for at least about 15 minutes after nanoparticle formation. In some embodiments, the nanoparticles have an average diameter of from about 10 nm to about 230 nm for at least about 15 minutes after nanoparticle formation. In some embodiments, the nanoparticles have an average diameter of from about 10 nm to about 220 nm for at least about 15 minutes after nanoparticle formation. In some embodiments, the nanoparticles have an average diameter of from about 10 nm to about 210 nm for at least about 15 minutes after nanoparticle formation. In some embodiments, the nanoparticles have an average diameter of from about 10 nm to about 200 nm for at least about 15 minutes after nanoparticle formation. In some embodiments, the nanoparticles have an average diameter of from about 10 nm to about 190 nm for at least about 15 minutes after nanoparticle formation. In some embodiments, the nanoparticles have an average diameter of from about 10 nm to about 180 nm for at least about 15 minutes after nanoparticle formation. In some embodiments, the nanoparticles have an average diameter of from about 10 nm to about 170 nm for at least about 15 minutes after nanoparticle formation. In some embodiments, the nanoparticles have an average diameter of from about 10 nm to about 160 nm for at least about 15 minutes after nanoparticle formation. In some embodiments, the nanoparticles have an average diameter of from about 10 nm to about 150 nm for at least about 15 minutes after nanoparticle formation. In some embodiments, the nanoparticles have an average diameter of from about 10 nm to about 140 nm for at least about 15 minutes after nanoparticle formation. In some embodiments, the nanoparticles have an average diameter of from about 10 nm to about 130 nm for at least about 15 minutes after nanoparticle formation. In some embodiments, the nanoparticles have an average diameter of from about 10 nm to about 120 nm for at least about 15 minutes after nanoparticle formation. In some embodiments, the nanoparticles have an average diameter of from about 10 nm to about 110 nm for at least about 15 minutes after nanoparticle formation. In some embodiments, the nanoparticles have an average diameter of from about 10 nm to about 100 nm for at least about 15 minutes after nanoparticle formation. In some embodiments, the nanoparticles have an average diameter of from about 10 nm to about 90 nm for at least about 15 minutes after nanoparticle formation. In some embodiments, the nanoparticles have an average diameter of from about 10 nm to about 80 nm for at least about 15 minutes after nanoparticle formation. In some embodiments, the nanoparticles have an average diameter of from about 10 nm to about 70 nm for at least about 15 minutes after nanoparticle formation. In some embodiments, the nanoparticles have an average diameter of from about 10 nm to about 60 nm for at least about 15 minutes after nanoparticle formation. In some embodiments, the nanoparticles have an average diameter of from about 10 nm to about 50 nm for at least about 15 minutes after nanoparticle formation. In some embodiments, the nanoparticles have an average diameter of from about 10 nm to about 40 nm for at least about 15 minutes after nanoparticle formation. In some embodiments, the nanoparticles have an average diameter of from about 10 nm to about 30 nm for at least about 15 minutes after nanoparticle formation. In some embodiments, the nanoparticles have an average diameter of from about 10 nm to about 20 nm for at least about 15 minutes after nanoparticle formation.

In some embodiments, the nanoparticles have an average diameter of about 10 nm for at least about 15 minutes after nanoparticle formation. In some embodiments, the nanoparticles have an average diameter of about 20 nm for at least about 15 minutes after nanoparticle formation. In some embodiments, the nanoparticles have an average diameter of about 30 nm for at least about 15 minutes after nanoparticle formation. In some embodiments, the nanoparticles have an average diameter of about 40 nm for at least about 15 minutes after nanoparticle formation. In some embodiments, the nanoparticles have an average diameter of about 50 nm for at least about 15 minutes after nanoparticle formation. In some embodiments, the nanoparticles have an average diameter of about 60 nm for at least about 15 minutes after nanoparticle formation. In some embodiments, the nanoparticles have an average diameter of about 70 nm for at least about 15 minutes after nanoparticle formation. In some embodiments, the nanoparticles have an average diameter of about 80 nm for at least about 15 minutes after nanoparticle formation. In some embodiments, the nanoparticles have an average diameter of about 90 nm for at least about 15 minutes after nanoparticle formation. In some embodiments, the nanoparticles have an average diameter of about 100 nm for at least about 15 minutes after nanoparticle formation. In some embodiments, the nanoparticles have an average diameter of about 110 nm for at least about 15 minutes after nanoparticle formation. In some embodiments, the nanoparticles have an average diameter of about 120 nm for at least about 15 minutes after nanoparticle formation. In some embodiments, the nanoparticles have an average diameter of about 130 nm for at least about 15 minutes after nanoparticle formation. In some embodiments, the nanoparticles have an average diameter of about 140 nm for at least about 15 minutes after nanoparticle formation. In some embodiments, the nanoparticles have an average diameter of about 150 nm for at least about 15 minutes after nanoparticle formation. In some embodiments, the nanoparticles have an average diameter of about 160 nm for at least about 15 minutes after nanoparticle formation. In some embodiments, the nanoparticles have an average diameter of about 170 nm for at least about 15 minutes after nanoparticle formation. In some embodiments, the nanoparticles have an average diameter of about 180 nm for at least about 15 minutes after nanoparticle formation. In some embodiments, the nanoparticles have an average diameter of about 190 nm for at least about 15 minutes after nanoparticle formation. In some embodiments, the nanoparticles have an average diameter of about 200 nm for at least about 15 minutes after nanoparticle formation. In some embodiments, the nanoparticles have an average diameter of about 210 nm for at least about 15 minutes after nanoparticle formation. In some embodiments, the nanoparticles have an average diameter of about 220 nm for at least about 15 minutes after nanoparticle formation. In some embodiments, the nanoparticles have an average diameter of about 230 nm for at least about 15 minutes after nanoparticle formation. In some embodiments, the nanoparticles have an average diameter of about 240 nm for at least about 15 minutes after nanoparticle formation. In some embodiments, the nanoparticles have an average diameter of about 250 nm for at least about 15 minutes after nanoparticle formation. In some embodiments, the nanoparticles have an average diameter of about 300 nm for at least about 15 minutes after nanoparticle formation. In some embodiments, the nanoparticles have an average diameter of about 350 nm for at least about 15 minutes after nanoparticle formation. In some embodiments, the nanoparticles have an average diameter of about 400 nm for at least about 15 minutes after nanoparticle formation. In some embodiments, the nanoparticles have an average diameter of about 450 nm for at least about 15 minutes after nanoparticle formation. In some embodiments, the nanoparticles have an average diameter of about 500 nm for at least about 15 minutes after nanoparticle formation. In some embodiments, the nanoparticles have an average diameter of about 550 nm for at least about 15 minutes after nanoparticle formation. In some embodiments, the nanoparticles have an average diameter of about 600 nm for at least about 15 minutes after nanoparticle formation. In some embodiments, the nanoparticles have an average diameter of about 650 nm for at least about 15 minutes after nanoparticle formation. In some embodiments, the nanoparticles have an average diameter of about 700 nm for at least about 15 minutes after nanoparticle formation. In some embodiments, the nanoparticles have an average diameter of about 750 nm for at least about 15 minutes after nanoparticle formation. In some embodiments, the nanoparticles have an average diameter of about 800 nm for at least about 15 minutes after nanoparticle formation. In some embodiments, the nanoparticles have an average diameter of about 850 nm for at least about 15 minutes after nanoparticle formation. In some embodiments, the nanoparticles have an average diameter of about 900 nm for at least about 15 minutes after nanoparticle formation. In some embodiments, the nanoparticles have an average diameter of about 950 nm for at least about 15 minutes after nanoparticle formation. In some embodiments, the nanoparticles have an average diameter of about 1000 nm for at least about 15 minutes after nanoparticle formation.

In some embodiments, the nanoparticles have an average diameter of about 1000 nm or less for at least about 2 hours after nanoparticle formation. In some embodiments, the nanoparticles have an average diameter of about 950 nm or less for at least about 2 hours after nanoparticle formation. In some embodiments, the nanoparticles have an average diameter of about 900 nm or less for at least about 2 hours after nanoparticle formation. In some embodiments, the nanoparticles have an average diameter of about 850 nm or less for at least about 2 hours after nanoparticle formation. In some embodiments, the nanoparticles have an average diameter of about 800 nm or less for at least about 2 hours after nanoparticle formation. In some embodiments, the nanoparticles have an average diameter of about 750 nm or less for at least about 2 hours after nanoparticle formation. In some embodiments, the nanoparticles have an average diameter of about 700 nm or less for at least about 2 hours after nanoparticle formation. In some embodiments, the nanoparticles have an average diameter of about 650 nm or less for at least about 2 hours after nanoparticle formation. In some embodiments, the nanoparticles have an average diameter of about 600 nm or less for at least about 2 hours after nanoparticle formation. In some embodiments, the nanoparticles have an average diameter of about 550 nm or less for at least about 2 hours after nanoparticle formation. In some embodiments, the nanoparticles have an average diameter of about 500 nm or less for at least about 2 hours after nanoparticle formation. In some embodiments, the nanoparticles have an average diameter of about 450 nm or less for at least about 2 hours after nanoparticle formation. In some embodiments, the nanoparticles have an average diameter of about 400 nm or less for at least about 2 hours after nanoparticle formation. In some embodiments, the nanoparticles have an average diameter of about 350 nm or less for at least about 2 hours after nanoparticle formation. In some embodiments, the nanoparticles have an average diameter of about 300 nm or less for at least about 2 hours after nanoparticle formation. In some embodiments, the nanoparticles have an average diameter of about 250 nm or less for at least about 2 hours after nanoparticle formation. In some embodiments, the nanoparticles have an average diameter of about 240 nm or less for at least about 2 hours after nanoparticle formation. In some embodiments, the nanoparticles have an average diameter of about 230 nm or less for at least about 2 hours after nanoparticle formation. In some embodiments, the nanoparticles have an average diameter of about 220 nm or less for at least about 2 hours after nanoparticle formation. In some embodiments, the nanoparticles have an average diameter of about 210 nm or less for at least about 2 hours after nanoparticle formation. In some embodiments, the nanoparticles have an average diameter of about 200 nm or less for at least about 2 hours after nanoparticle formation. In some embodiments, the nanoparticles have an average diameter of about 190 nm or less for at least about 2 hours after nanoparticle formation. In some embodiments, the nanoparticles have an average diameter of about 180 nm or less for at least about 2 hours after nanoparticle formation. In some embodiments, the nanoparticles have an average diameter of about 170 nm or less for at least about 2 hours after nanoparticle formation. In some embodiments, the nanoparticles have an average diameter of about 160 nm or less for at least about 2 hours after nanoparticle formation. In some embodiments, the nanoparticles have an average diameter of about 150 nm or less for at least about 2 hours after nanoparticle formation. In some embodiments, the nanoparticles have an average diameter of about 140 nm or less for at least about 2 hours after nanoparticle formation. In some embodiments, the nanoparticles have an average diameter of about 130 nm or less for at least about 2 hours after nanoparticle formation. In some embodiments, the nanoparticles have an average diameter of about 120 nm or less for at least about 2 hours after nanoparticle formation. In some embodiments, the nanoparticles have an average diameter of about 110 nm or less for at least about 2 hours after nanoparticle formation. In some embodiments, the nanoparticles have an average diameter of about 100 nm or less for at least about 2 hours after nanoparticle formation. In some embodiments, the nanoparticles have an average diameter of about 90 nm or less for at least about 2 hours after nanoparticle formation. In some embodiments, the nanoparticles have an average diameter of about 80 nm or less for at least about 2 hours after nanoparticle formation. In some embodiments, the nanoparticles have an average diameter of about 70 nm or less for at least about 2 hours after nanoparticle formation. In some embodiments, the nanoparticles have an average diameter of about 60 nm or less for at least about 2 hours after nanoparticle formation. In some embodiments, the nanoparticles have an average diameter of about 50 nm or less for at least about 2 hours after nanoparticle formation. In some embodiments, the nanoparticles have an average diameter of about 40 nm or less for at least about 2 hours after nanoparticle formation. In some embodiments, the nanoparticles have an average diameter of about 30 nm or less for at least about 2 hours after nanoparticle formation. In some embodiments, the nanoparticles have an average diameter of about 20 nm or less for at least about 2 hours after nanoparticle formation. In some embodiments, the nanoparticles have an average diameter of about 10 nm or less for at least about 2 hours after nanoparticle formation.

In some embodiments, the nanoparticles have an average diameter of about 10 nm or greater for at least about 2 hours after nanoparticle formation. In some embodiments, the nanoparticles have an average diameter of about 20 nm or greater for at least about 2 hours after nanoparticle formation. In some embodiments, the nanoparticles have an average diameter of about 30 nm or greater for at least about 2 hours after nanoparticle formation. In some embodiments, the nanoparticles have an average diameter of about 40 nm or greater for at least about 2 hours after nanoparticle formation. In some embodiments, the nanoparticles have an average diameter of about 50 nm or greater for at least about 2 hours after nanoparticle formation. In some embodiments, the nanoparticles have an average diameter of about 60 nm or greater for at least about 2 hours after nanoparticle formation. In some embodiments, the nanoparticles have an average diameter of about 70 nm or greater for at least about 2 hours after nanoparticle formation. In some embodiments, the nanoparticles have an average diameter of about 80 nm or greater for at least about 2 hours after nanoparticle formation. In some embodiments, the nanoparticles have an average diameter of about 90 nm or greater for at least about 2 hours after nanoparticle formation. In some embodiments, the nanoparticles have an average diameter of about 100 nm or greater for at least about 2 hours after nanoparticle formation. In some embodiments, the nanoparticles have an average diameter of about 110 nm or greater for at least about 2 hours after nanoparticle formation. In some embodiments, the nanoparticles have an average diameter of about 120 nm or greater for at least about 2 hours after nanoparticle formation. In some embodiments, the nanoparticles have an average diameter of about 130 nm or greater for at least about 2 hours after nanoparticle formation. In some embodiments, the nanoparticles have an average diameter of about 140 nm or greater for at least about 2 hours after nanoparticle formation. In some embodiments, the nanoparticles have an average diameter of about 150 nm or greater for at least about 2 hours after nanoparticle formation. In some embodiments, the nanoparticles have an average diameter of about 160 nm or greater for at least about 2 hours after nanoparticle formation. In some embodiments, the nanoparticles have an average diameter of about 170 nm or greater for at least about 2 hours after nanoparticle formation. In some embodiments, the nanoparticles have an average diameter of about 180 nm or greater for at least about 2 hours after nanoparticle formation. In some embodiments, the nanoparticles have an average diameter of about 190 nm or greater for at least about 2 hours after nanoparticle formation. In some embodiments, the nanoparticles have an average diameter of about 200 nm or greater for at least about 2 hours after nanoparticle formation. In some embodiments, the nanoparticles have an average diameter of about 210 nm or greater for at least about 2 hours after nanoparticle formation. In some embodiments, the nanoparticles have an average diameter of about 220 nm or greater for at least about 2 hours after nanoparticle formation. In some embodiments, the nanoparticles have an average diameter of about 230 nm or greater for at least about 2 hours after nanoparticle formation. In some embodiments, the nanoparticles have an average diameter of about 240 nm or greater for at least about 2 hours after nanoparticle formation. In some embodiments, the nanoparticles have an average diameter of about 250 nm or greater for at least about 2 hours after nanoparticle formation. In some embodiments, the nanoparticles have an average diameter of about 300 nm or greater for at least about 2 hours after nanoparticle formation. In some embodiments, the nanoparticles have an average diameter of about 350 nm or greater for at least about 2 hours after nanoparticle formation. In some embodiments, the nanoparticles have an average diameter of about 400 nm or greater for at least about 2 hours after nanoparticle formation. In some embodiments, the nanoparticles have an average diameter of about 450 nm or greater for at least about 2 hours after nanoparticle formation. In some embodiments, the nanoparticles have an average diameter of about 500 nm or greater for at least about 2 hours after nanoparticle formation. In some embodiments, the nanoparticles have an average diameter of about 550 nm or greater for at least about 2 hours after nanoparticle formation. In some embodiments, the nanoparticles have an average diameter of about 600 nm or greater for at least about 2 hours after nanoparticle formation. In some embodiments, the nanoparticles have an average diameter of about 650 nm or greater for at least about 2 hours after nanoparticle formation. In some embodiments, the nanoparticles have an average diameter of about 700 nm or greater for at least about 2 hours after nanoparticle formation. In some embodiments, the nanoparticles have an average diameter of about 750 nm or greater for at least about 2 hours after nanoparticle formation. In some embodiments, the nanoparticles have an average diameter of about 800 nm or greater for at least about 2 hours after nanoparticle formation. In some embodiments, the nanoparticles have an average diameter of about 850 nm or greater for at least about 2 hours after nanoparticle formation. In some embodiments, the nanoparticles have an average diameter of about 900 nm or greater for at least about 2 hours after nanoparticle formation. In some embodiments, the nanoparticles have an average diameter of about 950 nm or greater for at least about 2 hours after nanoparticle formation

In some embodiments, the nanoparticles have an average diameter of from about 10 nm to about 1000 nm for at least about 2 hours after nanoparticle formation. In some embodiments, the nanoparticles have an average diameter of from about 10 nm to about 950 nm for at least about 2 hours after nanoparticle formation. In some embodiments, the nanoparticles have an average diameter of from about 10 nm to about 900 nm for at least about 2 hours after nanoparticle formation. In some embodiments, the nanoparticles have an average diameter of from about 10 nm to about 850 nm for at least about 2 hours after nanoparticle formation. In some embodiments, the nanoparticles have an average diameter of from about 10 nm to about 800 nm for at least about 2 hours after nanoparticle formation. In some embodiments, the nanoparticles have an average diameter of from about 10 nm to about 750 nm for at least about 2 hours after nanoparticle formation. In some embodiments, the nanoparticles have an average diameter of from about 10 nm to about 700 nm for at least about 2 hours after nanoparticle formation. In some embodiments, the nanoparticles have an average diameter of from about 10 nm to about 650 nm for at least about 2 hours after nanoparticle formation. In some embodiments, the nanoparticles have an average diameter of from about 10 nm to about 600 nm for at least about 2 hours after nanoparticle formation. In some embodiments, the nanoparticles have an average diameter of from about 10 nm to about 550 nm for at least about 2 hours after nanoparticle formation for at least about 2 hours after nanoparticle formation. In some embodiments, the nanoparticles have an average diameter of from about 10 nm to about 500 nm for at least about 2 hours after nanoparticle formation. In some embodiments, the nanoparticles have an average diameter of from about 10 nm to about 450 nm for at least about 2 hours after nanoparticle formation. In some embodiments, the nanoparticles have an average diameter of from about 10 nm to about 400 nm for at least about 2 hours after nanoparticle formation. In some embodiments, the nanoparticles have an average diameter of from about 10 nm to about 350 nm for at least about 2 hours after nanoparticle formation. In some embodiments, the nanoparticles have an average diameter of from about 10 nm to about 300 nm for at least about 2 hours after nanoparticle formation. In some embodiments, the nanoparticles have an average diameter of from about 10 nm to about 250 nm for at least about 2 hours after nanoparticle formation. In some embodiments, the nanoparticles have an average diameter of from about 10 nm to about 240 nm for at least about 2 hours after nanoparticle formation. In some embodiments, the nanoparticles have an average diameter of from about 10 nm to about 230 nm for at least about 2 hours after nanoparticle formation. In some embodiments, the nanoparticles have an average diameter of from about 10 nm to about 220 nm for at least about 2 hours after nanoparticle formation. In some embodiments, the nanoparticles have an average diameter of from about 10 nm to about 210 nm for at least about 2 hours after nanoparticle formation. In some embodiments, the nanoparticles have an average diameter of from about 10 nm to about 200 nm for at least about 2 hours after nanoparticle formation. In some embodiments, the nanoparticles have an average diameter of from about 10 nm to about 190 nm for at least about 2 hours after nanoparticle formation. In some embodiments, the nanoparticles have an average diameter of from about 10 nm to about 180 nm for at least about 2 hours after nanoparticle formation. In some embodiments, the nanoparticles have an average diameter of from about 10 nm to about 170 nm for at least about 2 hours after nanoparticle formation. In some embodiments, the nanoparticles have an average diameter of from about 10 nm to about 160 nm for at least about 2 hours after nanoparticle formation. In some embodiments, the nanoparticles have an average diameter of from about 10 nm to about 150 nm for at least about 2 hours after nanoparticle formation. In some embodiments, the nanoparticles have an average diameter of from about 10 nm to about 140 nm for at least about 2 hours after nanoparticle formation. In some embodiments, the nanoparticles have an average diameter of from about 10 nm to about 130 nm for at least about 2 hours after nanoparticle formation. In some embodiments, the nanoparticles have an average diameter of from about 10 nm to about 120 nm for at least about 2 hours after nanoparticle formation. In some embodiments, the nanoparticles have an average diameter of from about 10 nm to about 110 nm for at least about 2 hours after nanoparticle formation. In some embodiments, the nanoparticles have an average diameter of from about 10 nm to about 100 nm for at least about 2 hours after nanoparticle formation. In some embodiments, the nanoparticles have an average diameter of from about 10 nm to about 90 nm for at least about 2 hours after nanoparticle formation. In some embodiments, the nanoparticles have an average diameter of from about 10 nm to about 80 nm for at least about 2 hours after nanoparticle formation. In some embodiments, the nanoparticles have an average diameter of from about 10 nm to about 70 nm for at least about 2 hours after nanoparticle formation. In some embodiments, the nanoparticles have an average diameter of from about 10 nm to about 60 nm for at least about 2 hours after nanoparticle formation. In some embodiments, the nanoparticles have an average diameter of from about 10 nm to about 50 nm for at least about 2 hours after nanoparticle formation. In some embodiments, the nanoparticles have an average diameter of from about 10 nm to about 40 nm for at least about 2 hours after nanoparticle formation. In some embodiments, the nanoparticles have an average diameter of from about 10 nm to about 30 nm for at least about 2 hours after nanoparticle formation. In some embodiments, the nanoparticles have an average diameter of from about 10 nm to about 20 nm for at least about 2 hours after nanoparticle formation.

In some embodiments, the nanoparticles have an average diameter of about 10 nm for at least about 2 hours after nanoparticle formation. In some embodiments, the nanoparticles have an average diameter of about 20 nm for at least about 2 hours after nanoparticle formation. In some embodiments, the nanoparticles have an average diameter of about 30 nm for at least about 2 hours after nanoparticle formation. In some embodiments, the nanoparticles have an average diameter of about 40 nm for at least about 2 hours after nanoparticle formation. In some embodiments, the nanoparticles have an average diameter of about 50 nm for at least about 2 hours after nanoparticle formation. In some embodiments, the nanoparticles have an average diameter of about 60 nm for at least about 2 hours after nanoparticle formation. In some embodiments, the nanoparticles have an average diameter of about 70 nm for at least about 2 hours after nanoparticle formation. In some embodiments, the nanoparticles have an average diameter of about 80 nm for at least about 2 hours after nanoparticle formation. In some embodiments, the nanoparticles have an average diameter of about 90 nm for at least about 2 hours after nanoparticle formation. In some embodiments, the nanoparticles have an average diameter of about 100 nm for at least about 2 hours after nanoparticle formation. In some embodiments, the nanoparticles have an average diameter of about 110 nm for at least about 2 hours after nanoparticle formation. In some embodiments, the nanoparticles have an average diameter of about 120 nm for at least about 2 hours after nanoparticle formation. In some embodiments, the nanoparticles have an average diameter of about 130 nm for at least about 2 hours after nanoparticle formation. In some embodiments, the nanoparticles have an average diameter of about 140 nm for at least about 2 hours after nanoparticle formation. In some embodiments, the nanoparticles have an average diameter of about 150 nm for at least about 2 hours after nanoparticle formation. In some embodiments, the nanoparticles have an average diameter of about 160 nm for at least about 2 hours after nanoparticle formation. In some embodiments, the nanoparticles have an average diameter of about 170 nm for at least about 2 hours after nanoparticle formation. In some embodiments, the nanoparticles have an average diameter of about 180 nm for at least about 2 hours after nanoparticle formation. In some embodiments, the nanoparticles have an average diameter of about 190 nm for at least about 2 hours after nanoparticle formation. In some embodiments, the nanoparticles have an average diameter of about 200 nm for at least about 2 hours after nanoparticle formation. In some embodiments, the nanoparticles have an average diameter of about 210 nm for at least about 2 hours after nanoparticle formation. In some embodiments, the nanoparticles have an average diameter of about 220 nm for at least about 2 hours after nanoparticle formation. In some embodiments, the nanoparticles have an average diameter of about 230 nm for at least about 2 hours after nanoparticle formation. In some embodiments, the nanoparticles have an average diameter of about 240 nm for at least about 2 hours after nanoparticle formation. In some embodiments, the nanoparticles have an average diameter of about 250 nm for at least about 2 hours after nanoparticle formation. In some embodiments, the nanoparticles have an average diameter of about 300 nm for at least about 2 hours after nanoparticle formation. In some embodiments, the nanoparticles have an average diameter of about 350 nm for at least about 2 hours after nanoparticle formation. In some embodiments, the nanoparticles have an average diameter of about 400 nm for at least about 2 hours after nanoparticle formation. In some embodiments, the nanoparticles have an average diameter of about 450 nm for at least about 2 hours after nanoparticle formation. In some embodiments, the nanoparticles have an average diameter of about 500 nm for at least about 2 hours after nanoparticle formation. In some embodiments, the nanoparticles have an average diameter of about 550 nm for at least about 2 hours after nanoparticle formation. In some embodiments, the nanoparticles have an average diameter of about 600 nm for at least about 2 hours after nanoparticle formation. In some embodiments, the nanoparticles have an average diameter of about 650 nm for at least about 2 hours after nanoparticle formation. In some embodiments, the nanoparticles have an average diameter of about 700 nm for at least about 2 hours after nanoparticle formation. In some embodiments, the nanoparticles have an average diameter of about 750 nm for at least about 2 hours after nanoparticle formation. In some embodiments, the nanoparticles have an average diameter of about 800 nm for at least about 2 hours after nanoparticle formation. In some embodiments, the nanoparticles have an average diameter of about 850 nm for at least about 2 hours after nanoparticle formation. In some embodiments, the nanoparticles have an average diameter of about 900 nm for at least about 2 hours after nanoparticle formation. In some embodiments, the nanoparticles have an average diameter of about 950 nm for at least about 2 hours after nanoparticle formation. In some embodiments, the nanoparticles have an average diameter of about 1000 nm for at least about 2 hours after nanoparticle formation.

In some embodiments, the nanoparticles have an average diameter of from about 10 nm to about 1000 nm. In some embodiments, the nanoparticles have an average diameter of from about 10 nm to about 950 nm. In some embodiments, the nanoparticles have an average diameter of from about 10 nm to about 900 nm. In some embodiments, the nanoparticles have an average diameter of from about 10 nm to about 850 nm. In some embodiments, the nanoparticles have an average diameter of from about 10 nm to about 800 nm. In some embodiments, the nanoparticles have an average diameter of from about 10 nm to about 750 nm. In some embodiments, the nanoparticles have an average diameter of from about 10 nm to about 700 nm. In some embodiments, the nanoparticles have an average diameter of from about 10 nm to about 650 nm. In some embodiments, the nanoparticles have an average diameter of from about 10 nm to about 600 nm. In some embodiments, the nanoparticles have an average diameter of from about 10 nm to about 550 nm. In some embodiments, the nanoparticles have an average diameter of from about 10 nm to about 500 nm. In some embodiments, the nanoparticles have an average diameter of from about 10 nm to about 450 nm. In some embodiments, the nanoparticles have an average diameter of from about 10 nm to about 400 nm. In some embodiments, the nanoparticles have an average diameter of from about 10 nm to about 350 nm. In some embodiments, the nanoparticles have an average diameter of from about 10 nm to about 300 nm. In some embodiments, the nanoparticles have an average diameter of from about 10 nm to about 250 nm. In some embodiments, the nanoparticles have an average diameter of from about 10 nm to about 240 nm. In some embodiments, the nanoparticles have an average diameter of from about 10 nm to about 230 nm. In some embodiments, the nanoparticles have an average diameter of from about 10 nm to about 220 nm. In some embodiments, the nanoparticles have an average diameter of from about 10 nm to about 210 nm. In some embodiments, the nanoparticles have an average diameter of from about 10 nm to about 200 nm. In some embodiments, the nanoparticles have an average diameter of from about 10 nm to about 190 nm. In some embodiments, the nanoparticles have an average diameter of from about 10 nm to about 180 nm. In some embodiments, the nanoparticles have an average diameter of from about 10 nm to about 170 nm. In some embodiments, the nanoparticles have an average diameter of from about 10 nm to about 160 nm. In some embodiments, the nanoparticles have an average diameter of from about 10 nm to about 150 nm. In some embodiments, the nanoparticles have an average diameter of from about 10 nm to about 140 nm. In some embodiments, the nanoparticles have an average diameter of from about 10 nm to about 130 nm. In some embodiments, the nanoparticles have an average diameter of from about 10 nm to about 120 nm. In some embodiments, the nanoparticles have an average diameter of from about 10 nm to about 110 nm. In some embodiments, the nanoparticles have an average diameter of from about 10 nm to about 100 nm. In some embodiments, the nanoparticles have an average diameter of from about 10 nm to about 90 nm. In some embodiments, the nanoparticles have an average diameter of from about 10 nm to about 80 nm. In some embodiments, the nanoparticles have an average diameter of from about 10 nm to about 70 nm. In some embodiments, the nanoparticles have an average diameter of from about 10 nm to about 60 nm. In some embodiments, the nanoparticles have an average diameter of from about 10 nm to about 50 nm. In some embodiments, the nanoparticles have an average diameter of from about 10 nm to about 40 nm. In some embodiments, the nanoparticles have an average diameter of from about 10 nm to about 30 nm. In some embodiments, the nanoparticles have an average diameter of from about 10 nm to about 20 nm.

In some embodiments, the nanoparticles have an average diameter of from about 20 nm to about 1000 nm. In some embodiments, the nanoparticles have an average diameter of from about 20 nm to about 950 nm. In some embodiments, the nanoparticles have an average diameter of from about 20 nm to about 900 nm. In some embodiments, the nanoparticles have an average diameter of from about 20 nm to about 850 nm. In some embodiments, the nanoparticles have an average diameter of from about 20 nm to about 800 nm. In some embodiments, the nanoparticles have an average diameter of from about 20 nm to about 750 nm. In some embodiments, the nanoparticles have an average diameter of from about 20 nm to about 700 nm. In some embodiments, the nanoparticles have an average diameter of from about 20 nm to about 650 nm. In some embodiments, the nanoparticles have an average diameter of from about 20 nm to about 600 nm. In some embodiments, the nanoparticles have an average diameter of from about 20 nm to about 550 nm. In some embodiments, the nanoparticles have an average diameter of from about 20 nm to about 500 nm. In some embodiments, the nanoparticles have an average diameter of from about 20 nm to about 450 nm. In some embodiments, the nanoparticles have an average diameter of from about 20 nm to about 400 nm. In some embodiments, the nanoparticles have an average diameter of from about 20 nm to about 350 nm. In some embodiments, the nanoparticles have an average diameter of from about 20 nm to about 300 nm. In some embodiments, the nanoparticles have an average diameter of from about 20 nm to about 250 nm. In some embodiments, the nanoparticles have an average diameter of from about 20 nm to about 240 nm. In some embodiments, the nanoparticles have an average diameter of from about 20 nm to about 230 nm. In some embodiments, the nanoparticles have an average diameter of from about 20 nm to about 220 nm. In some embodiments, the nanoparticles have an average diameter of from about 20 nm to about 210 nm. In some embodiments, the nanoparticles have an average diameter of from about 20 nm to about 200 nm. In some embodiments, the nanoparticles have an average diameter of from about 20 nm to about 190 nm. In some embodiments, the nanoparticles have an average diameter of from about 20 nm to about 180 nm. In some embodiments, the nanoparticles have an average diameter of from about 20 nm to about 170 nm. In some embodiments, the nanoparticles have an average diameter of from about 20 nm to about 160 nm. In some embodiments, the nanoparticles have an average diameter of from about 20 nm to about 150 nm. In some embodiments, the nanoparticles have an average diameter of from about 20 nm to about 140 nm. In some embodiments, the nanoparticles have an average diameter of from about 20 nm to about 130 nm. In some embodiments, the nanoparticles have an average diameter of from about 20 nm to about 120 nm. In some embodiments, the nanoparticles have an average diameter of from about 20 nm to about 110 nm. In some embodiments, the nanoparticles have an average diameter of from about 20 nm to about 100 nm. In some embodiments, the nanoparticles have an average diameter of from about 20 nm to about 90 nm. In some embodiments, the nanoparticles have an average diameter of from about 20 nm to about 80 nm. In some embodiments, the nanoparticles have an average diameter of from about 20 nm to about 70 nm. In some embodiments, the nanoparticles have an average diameter of from about 20 nm to about 60 nm. In some embodiments, the nanoparticles have an average diameter of from about 20 nm to about 50 nm. In some embodiments, the nanoparticles have an average diameter of from about 20 nm to about 40 nm. In some embodiments, the nanoparticles have an average diameter of from about 20 nm to about 30 nm.

In some embodiments, the nanoparticles have an average diameter of from about 30 nm to about 1000 nm. In some embodiments, the nanoparticles have an average diameter of from about 30 nm to about 950 nm. In some embodiments, the nanoparticles have an average diameter of from about 30 nm to about 900 nm. In some embodiments, the nanoparticles have an average diameter of from about 30 nm to about 850 nm. In some embodiments, the nanoparticles have an average diameter of from about 30 nm to about 800 nm. In some embodiments, the nanoparticles have an average diameter of from about 30 nm to about 750 nm. In some embodiments, the nanoparticles have an average diameter of from about 30 nm to about 700 nm. In some embodiments, the nanoparticles have an average diameter of from about 30 nm to about 650 nm. In some embodiments, the nanoparticles have an average diameter of from about 30 nm to about 600 nm. In some embodiments, the nanoparticles have an average diameter of from about 30 nm to about 550 nm. In some embodiments, the nanoparticles have an average diameter of from about 30 nm to about 500 nm. In some embodiments, the nanoparticles have an average diameter of from about 30 nm to about 450 nm. In some embodiments, the nanoparticles have an average diameter of from about 30 nm to about 400 nm. In some embodiments, the nanoparticles have an average diameter of from about 30 nm to about 350 nm. In some embodiments, the nanoparticles have an average diameter of from about 30 nm to about 300 nm. In some embodiments, the nanoparticles have an average diameter of from about 30 nm to about 250 nm. In some embodiments, the nanoparticles have an average diameter of from about 30 nm to about 240 nm. In some embodiments, the nanoparticles have an average diameter of from about 30 nm to about 230 nm. In some embodiments, the nanoparticles have an average diameter of from about 30 nm to about 220 nm. In some embodiments, the nanoparticles have an average diameter of from about 30 nm to about 210 nm. In some embodiments, the nanoparticles have an average diameter of from about 30 nm to about 200 nm. In some embodiments, the nanoparticles have an average diameter of from about 30 nm to about 190 nm. In some embodiments, the nanoparticles have an average diameter of from about 30 nm to about 180 nm. In some embodiments, the nanoparticles have an average diameter of from about 30 nm to about 170 nm. In some embodiments, the nanoparticles have an average diameter of from about 30 nm to about 160 nm. In some embodiments, the nanoparticles have an average diameter of from about 30 nm to about 150 nm. In some embodiments, the nanoparticles have an average diameter of from about 30 nm to about 140 nm. In some embodiments, the nanoparticles have an average diameter of from about 30 nm to about 130 nm. In some embodiments, the nanoparticles have an average diameter of from about 30 nm to about 120 nm. In some embodiments, the nanoparticles have an average diameter of from about 30 nm to about 110 nm. In some embodiments, the nanoparticles have an average diameter of from about 30 nm to about 100 nm. In some embodiments, the nanoparticles have an average diameter of from about 30 nm to about 90 nm. In some embodiments, the nanoparticles have an average diameter of from about 30 nm to about 80 nm. In some embodiments, the nanoparticles have an average diameter of from about 30 nm to about 70 nm. In some embodiments, the nanoparticles have an average diameter of from about 30 nm to about 60 nm. In some embodiments, the nanoparticles have an average diameter of from about 30 nm to about 50 nm. In some embodiments, the nanoparticles have an average diameter of from about 30 nm to about 40 nm. In some embodiments, the nanoparticles have an average diameter of from about 30 nm to about 40 nm.

In some embodiments, the nanoparticles have an average diameter of from about 40 nm to about 1000 nm. In some embodiments, the nanoparticles have an average diameter of from about 40 nm to about 950 nm. In some embodiments, the nanoparticles have an average diameter of from about 40 nm to about 900 nm. In some embodiments, the nanoparticles have an average diameter of from about 40 nm to about 850 nm. In some embodiments, the nanoparticles have an average diameter of from about 40 nm to about 800 nm. In some embodiments, the nanoparticles have an average diameter of from about 40 nm to about 750 nm. In some embodiments, the nanoparticles have an average diameter of from about 40 nm to about 700 nm. In some embodiments, the nanoparticles have an average diameter of from about 40 nm to about 650 nm. In some embodiments, the nanoparticles have an average diameter of from about 40 nm to about 600 nm. In some embodiments, the nanoparticles have an average diameter of from about 40 nm to about 550 nm. In some embodiments, the nanoparticles have an average diameter of from about 40 nm to about 500 nm. In some embodiments, the nanoparticles have an average diameter of from about 40 nm to about 450 nm. In some embodiments, the nanoparticles have an average diameter of from about 40 nm to about 400 nm. In some embodiments, the nanoparticles have an average diameter of from about 40 nm to about 350 nm. In some embodiments, the nanoparticles have an average diameter of from about 40 nm to about 300 nm. In some embodiments, the nanoparticles have an average diameter of from about 40 nm to about 250 nm. In some embodiments, the nanoparticles have an average diameter of from about 40 nm to about 240 nm. In some embodiments, the nanoparticles have an average diameter of from about 40 nm to about 230 nm. In some embodiments, the nanoparticles have an average diameter of from about 40 nm to about 220 nm. In some embodiments, the nanoparticles have an average diameter of from about 40 nm to about 210 nm. In some embodiments, the nanoparticles have an average diameter of from about 40 nm to about 200 nm. In some embodiments, the nanoparticles have an average diameter of from about 40 nm to about 190 nm. In some embodiments, the nanoparticles have an average diameter of from about 40 nm to about 180 nm. In some embodiments, the nanoparticles have an average diameter of from about 40 nm to about 170 nm. In some embodiments, the nanoparticles have an average diameter of from about 40 nm to about 160 nm. In some embodiments, the nanoparticles have an average diameter of from about 40 nm to about 150 nm. In some embodiments, the nanoparticles have an average diameter of from about 40 nm to about 140 nm. In some embodiments, the nanoparticles have an average diameter of from about 40 nm to about 130 nm. In some embodiments, the nanoparticles have an average diameter of from about 40 nm to about 120 nm. In some embodiments, the nanoparticles have an average diameter of from about 40 nm to about 110 nm. In some embodiments, the nanoparticles have an average diameter of from about 40 nm to about 100 nm. In some embodiments, the nanoparticles have an average diameter of from about 40 nm to about 90 nm. In some embodiments, the nanoparticles have an average diameter of from about 40 nm to about 80 nm. In some embodiments, the nanoparticles have an average diameter of from about 40 nm to about 70 nm. In some embodiments, the nanoparticles have an average diameter of from about 40 nm to about 60 nm. In some embodiments, the nanoparticles have an average diameter of from about 40 nm to about 50 nm.

In some embodiments, the nanoparticles have an average diameter of from about 50 nm to about 1000 nm. In some embodiments, the nanoparticles have an average diameter of from about 50 nm to about 950 nm. In some embodiments, the nanoparticles have an average diameter of from about 50 nm to about 900 nm. In some embodiments, the nanoparticles have an average diameter of from about 50 nm to about 850 nm. In some embodiments, the nanoparticles have an average diameter of from about 50 nm to about 800 nm. In some embodiments, the nanoparticles have an average diameter of from about 50 nm to about 750 nm. In some embodiments, the nanoparticles have an average diameter of from about 50 nm to about 700 nm. In some embodiments, the nanoparticles have an average diameter of from about 50 nm to about 650 nm. In some embodiments, the nanoparticles have an average diameter of from about 50 nm to about 600 nm. In some embodiments, the nanoparticles have an average diameter of from about 50 nm to about 550 nm. In some embodiments, the nanoparticles have an average diameter of from about 50 nm to about 500 nm. In some embodiments, the nanoparticles have an average diameter of from about 50 nm to about 450 nm. In some embodiments, the nanoparticles have an average diameter of from about 50 nm to about 400 nm. In some embodiments, the nanoparticles have an average diameter of from about 50 nm to about 350 nm. In some embodiments, the nanoparticles have an average diameter of from about 50 nm to about 300 nm. In some embodiments, the nanoparticles have an average diameter of from about 50 nm to about 250 nm. In some embodiments, the nanoparticles have an average diameter of from about 50 nm to about 240 nm. In some embodiments, the nanoparticles have an average diameter of from about 50 nm to about 230 nm. In some embodiments, the nanoparticles have an average diameter of from about 50 nm to about 220 nm. In some embodiments, the nanoparticles have an average diameter of from about 50 nm to about 210 nm. In some embodiments, the nanoparticles have an average diameter of from about 50 nm to about 200 nm. In some embodiments, the nanoparticles have an average diameter of from about 50 nm to about 190 nm. In some embodiments, the nanoparticles have an average diameter of from about 50 nm to about 180 nm. In some embodiments, the nanoparticles have an average diameter of from about 50 nm to about 170 nm. In some embodiments, the nanoparticles have an average diameter of from about 50 nm to about 160 nm. In some embodiments, the nanoparticles have an average diameter of from about 50 nm to about 150 nm. In some embodiments, the nanoparticles have an average diameter of from about 50 nm to about 140 nm. In some embodiments, the nanoparticles have an average diameter of from about 50 nm to about 130 nm. In some embodiments, the nanoparticles have an average diameter of from about 50 nm to about 120 nm. In some embodiments, the nanoparticles have an average diameter of from about 50 nm to about 110 nm. In some embodiments, the nanoparticles have an average diameter of from about 50 nm to about 100 nm. In some embodiments, the nanoparticles have an average diameter of from about 50 nm to about 90 nm. In some embodiments, the nanoparticles have an average diameter of from about 50 nm to about 80 nm. In some embodiments, the nanoparticles have an average diameter of from about 50 nm to about 70 nm. In some embodiments, the nanoparticles have an average diameter of from about 50 nm to about 60 nm.

In some embodiments, the nanoparticles have an average diameter of about 10 nm. In some embodiments, the nanoparticles have an average diameter of about 20 nm. In some embodiments, the nanoparticles have an average diameter of about 30 nm. In some embodiments, the nanoparticles have an average diameter of about 40 nm. In some embodiments, the nanoparticles have an average diameter of about 50 nm. In some embodiments, the nanoparticles have an average diameter of about 60 nm. In some embodiments, the nanoparticles have an average diameter of about 70 nm. In some embodiments, the nanoparticles have an average diameter of about 80 nm. In some embodiments, the nanoparticles have an average diameter of about 90 nm. In some embodiments, the nanoparticles have an average diameter of about 100 nm. In some embodiments, the nanoparticles have an average diameter of about 110 nm. In some embodiments, the nanoparticles have an average diameter of about 120 nm. In some embodiments, the nanoparticles have an average diameter of about 130 nm. In some embodiments, the nanoparticles have an average diameter of about 140 nm. In some embodiments, the nanoparticles have an average diameter of about 150 nm. In some embodiments, the nanoparticles have an average diameter of about 160 nm. In some embodiments, the nanoparticles have an average diameter of about 170 nm. In some embodiments, the nanoparticles have an average diameter of about 180 nm. In some embodiments, the nanoparticles have an average diameter of about 190 nm. In some embodiments, the nanoparticles have an average diameter of about 200 nm. In some embodiments, the nanoparticles have an average diameter of about 210 nm. In some embodiments, the nanoparticles have an average diameter of about 220 nm. In some embodiments, the nanoparticles have an average diameter of about 230 nm. In some embodiments, the nanoparticles have an average diameter of about 240 nm. In some embodiments, the nanoparticles have an average diameter of about 250 nm. In some embodiments, the nanoparticles have an average diameter of about 300 nm. In some embodiments, the nanoparticles have an average diameter of about 350 nm. In some embodiments, the nanoparticles have an average diameter of about 400 nm. In some embodiments, the nanoparticles have an average diameter of about 450 nm. In some embodiments, the nanoparticles have an average diameter of about 500 nm. In some embodiments, the nanoparticles have an average diameter of about 550 nm. In some embodiments, the nanoparticles have an average diameter of about 600 nm. In some embodiments, the nanoparticles have an average diameter of about 650 nm. In some embodiments, the nanoparticles have an average diameter of about 700 nm. In some embodiments, the nanoparticles have an average diameter of about 750 nm. In some embodiments, the nanoparticles have an average diameter of about 800 nm. In some embodiments, the nanoparticles have an average diameter of about 850 nm. In some embodiments, the nanoparticles have an average diameter of about 900 nm. In some embodiments, the nanoparticles have an average diameter of about 950 nm. In some embodiments, the nanoparticles have an average diameter of about 1000 nm.

In some embodiments, the composition is sterile filterable. In some embodiments, the nanoparticles have an average diameter of about 250 nm or less. In some embodiments, the nanoparticles have an average diameter of about 240 nm or less. In some embodiments, the nanoparticles have an average diameter of about 230 nm or less. In some embodiments, the nanoparticles have an average diameter of about 220 nm or less. In some embodiments, the nanoparticles have an average diameter of about 210 nm or less. In some embodiments, the nanoparticles have an average diameter of about 200 nm or less. In some embodiments, the nanoparticles have an average diameter of from about 10 nm to about 250 nm. In some embodiments, the nanoparticles have an average diameter of from about 10 nm to about 240 nm. In some embodiments, the nanoparticles have an average diameter of from about 10 nm to about 230 nm. In some embodiments, the nanoparticles have an average diameter of from about 10 nm to about 220 nm. In some embodiments, the nanoparticles have an average diameter of from about 10 nm to about 210 nm. In some embodiments, the nanoparticles have an average diameter of from about 10 nm to about 200 nm.

In some embodiments, the nanoparticles are suspended, dissolved, or emulsified in a liquid. In some embodiments, the nanoparticles are suspended in a liquid. In some embodiments, the nanoparticles are dissolved in a liquid. In some embodiments, the nanoparticles are emulsified in a liquid.

### Dehydrated Composition

In some embodiments, the composition is dehydrated. In some embodiments, the composition is a lyophilized composition. In some embodiments, the dehydrated composition comprises less than about 10%, about 5%, about 4%, about 3%, about 2%, about 1%, about 0.9%, about 0.8%, about 0.7%, about 0.6%, about 0.5%, about 0.4%, about 0.3%, about 0.2%, about 0.1%, about 0.05%, or about 0.01% by weight of water. In some embodiments, the dehydrated composition comprises less than about 5%, about 4%, about 3%, about 2%, about 1%, about 0.9%, about 0.8%, about 0.7%, about 0.6%, about 0.5%, about 0.4%, about 0.3%, about 0.2%, about 0.1%, about 0.05%, or about 0.01% by weight of water.

In some embodiments, when the composition is dehydrated composition, such as a lyophilized composition, the composition comprises from about 0.1% to about 99% by weight of the compound. In some embodiments, the composition comprises from about 0.1% to about 75% by weight of the compound. In some embodiments, the composition comprises from about 0.1% to about 50% by weight of the compound. In some embodiments, the composition comprises from about 0.1% to about 25% by weight of the compound. In some embodiments, the composition comprises from about 0.1% to about 20% by weight of the compound. In some embodiments, the composition comprises from about 0.1% to about 15% by weight of the compound. In some embodiments, the composition comprises from about 0.1% to about 10% by weight of the compound.

In some embodiments, when the composition is dehydrated composition, such as a lyophilized composition, the composition comprises from about 0.5% to about 99% by weight of the compound. In some embodiments, the composition comprises from about 0.5% to about 75% by weight of the compound. In some embodiments, the composition comprises from about 0.5% to about 50% by weight of the compound. In some embodiments, the composition comprises from about 0.5% to about 25% by weight of the compound. In some embodiments, the composition comprises from about 0.5% to about 20% by weight of the compound. In some embodiments, the composition comprises from about 0.5% to about 15% by weight of the compound. In some embodiments, the composition comprises from about 0.5% to about 10% by weight of the compound.

In some embodiments, when the composition is dehydrated composition, such as a lyophilized composition, the composition comprises from about 0.9% to about 24% by weight of the compound. In some embodiments, the composition comprises from about 1.8% to about 16% by weight of the compound.

In some embodiments, when the composition is dehydrated composition, such as a lyophilized composition, the composition comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% by weight of the compound. In some embodiments, the composition comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of the compound. In some embodiments, the composition comprises about 0.9%, about 1%, about 1.1%, about 1.2%, about 1.3%, about 1.4%, about 1.5%, about 1.6%, about 1.7%, about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, or about 24% by weight of the compound. In some embodiments, the composition comprises about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, or about 16% by weight of the compound.

In some embodiments, when the composition is dehydrated composition, such as a lyophilized composition, the composition comprises from about 50% to about 99% by weight of the pharmaceutically acceptable carrier. In some embodiments, the composition comprises from about 55% to about 99% by weight of the pharmaceutically acceptable carrier. In some embodiments, the composition comprises from about 60% to about 99% by weight of the pharmaceutically acceptable carrier. In some embodiments, the composition comprises from about 65% to about 99% by weight of the pharmaceutically acceptable carrier. In some embodiments, the composition comprises from about 70% to about 99% by weight of the pharmaceutically acceptable carrier. In some embodiments, the composition comprises from about 75% to about 99% by weight of the pharmaceutically acceptable carrier. In some embodiments, the composition comprises from about 80% to about 99% by weight of the pharmaceutically acceptable carrier. In some embodiments, the composition comprises from about 85% to about 99% by weight of the pharmaceutically acceptable carrier. In some embodiments, the composition comprises from about 90% to about 99% by weight of the pharmaceutically acceptable carrier.

In some embodiments, when the composition is dehydrated composition, such as a lyophilized composition, the composition comprises from about 76% to about 99% by weight of the pharmaceutically acceptable carrier. In some embodiments, the composition comprises from about 84% to about 98% by weight of the pharmaceutically acceptable carrier.

In some embodiments, when the composition is dehydrated composition, such as a lyophilized composition, the composition comprises about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the pharmaceutically acceptable carrier. In some embodiments, the composition comprises about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the pharmaceutically acceptable carrier. In some embodiments, the composition comprises about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the pharmaceutically acceptable carrier.

### Reconstitution

In some embodiments, the composition is reconstituted with an appropriate biocompatible liquid to provide a reconstituted composition. In some embodiments, appropriate biocompatible liquid is a buffered solution. Examples of suitable buffered solutions include, but are not limited to, buffered solutions of amino acids, buffered solutions of proteins, buffered solutions of sugars, buffered solutions of vitamins, buffered solutions of synthetic polymers, buffered solutions of salts (such as buffered saline or buffered aqueous media), any similar buffered solutions, or any suitable combination thereof. In some embodiments, the appropriate biocompatible liquid is a solution comprising dextrose. In some embodiments, the appropriate biocompatible liquid is a solution comprising one or more salts. In some embodiments, the appropriate biocompatible liquid is a solution suitable for intravenous use. Examples of solutions that are suitable for intravenous use, include, but are not limited to, balanced solutions, which are different solutions with different electrolyte compositions that are close to plasma composition. Such electrolyte compositions comprise crystalloids or colloids. Examples of suitable appropriate biocompatible liquids include, but are not limited to, sterile water, saline, phosphate-buffered saline, 5% dextrose in water solution, Ringer's solution, or Ringer's lactate solution. In some embodiments, the appropriate biocompatible liquid is sterile water, saline, phosphate-buffered saline, 5% dextrose in water solution, Ringer's solution, or Ringer's lactate solution. In some embodiments, the appropriate biocompatible liquid is sterile water. In some embodiments, the appropriate biocompatible liquid is saline. In some embodiments, the appropriate biocompatible liquid is phosphate-buffered saline. In some embodiments, the appropriate biocompatible liquid is 5% dextrose in water solution. In some embodiments, the appropriate biocompatible liquid is Ringer's solution. In some embodiments, the appropriate biocompatible liquid is Ringer's lactate solution. In some embodiments, the appropriate biocompatible liquid is a balanced solution, or a solution with an electrolyte composition that resembles plasma.

In some embodiments, the nanoparticles have an average diameter of from about 10 nm to about 1000 nm after reconstitution. In some embodiments, the nanoparticles have an average diameter of from about 10 nm to about 950 nm after reconstitution. In some embodiments, the nanoparticles have an average diameter of from about 10 nm to about 900 nm after reconstitution. In some embodiments, the nanoparticles have an average diameter of from about 10 nm to about 850 nm after reconstitution. In some embodiments, the nanoparticles have an average diameter of from about 10 nm to about 800 nm after reconstitution. In some embodiments, the nanoparticles have an average diameter of from about 10 nm to about 750 nm after reconstitution. In some embodiments, the nanoparticles have an average diameter of from about 10 nm to about 700 nm after reconstitution. In some embodiments, the nanoparticles have an average diameter of from about 10 nm to about 650 nm after reconstitution. In some embodiments, the nanoparticles have an average diameter of from about 10 nm to about 600 nm after reconstitution. In some embodiments, the nanoparticles have an average diameter of from about 10 nm to about 550 nm after reconstitution. In some embodiments, the nanoparticles have an average diameter of from about 10 nm to about 500 nm after reconstitution. In some embodiments, the nanoparticles have an average diameter of from about 10 nm to about 450 nm after reconstitution. In some embodiments, the nanoparticles have an average diameter of from about 10 nm to about 400 nm after reconstitution. In some embodiments, the nanoparticles have an average diameter of from about 10 nm to about 350 nm after reconstitution. In some embodiments, the nanoparticles have an average diameter of from about 10 nm to about 300 nm after reconstitution. In some embodiments, the nanoparticles have an average diameter of from about 10 nm to about 250 nm after reconstitution. In some embodiments, the nanoparticles have an average diameter of from about 10 nm to about 240 nm after reconstitution. In some embodiments, the nanoparticles have an average diameter of from about 10 nm to about 230 nm after reconstitution. In some embodiments, the nanoparticles have an average diameter of from about 10 nm to about 220 nm after reconstitution. In some embodiments, the nanoparticles have an average diameter of from about 10 nm to about 210 nm after reconstitution. In some embodiments, the nanoparticles have an average diameter of from about 10 nm to about 200 nm after reconstitution. In some embodiments, the nanoparticles have an average diameter of from about 10 nm to about 190 nm after reconstitution. In some embodiments, the nanoparticles have an average diameter of from about 10 nm to about 180 nm after reconstitution. In some embodiments, the nanoparticles have an average diameter of from about 10 nm to about 170 nm after reconstitution. In some embodiments, the nanoparticles have an average diameter of from about 10 nm to about 160 nm after reconstitution. In some embodiments, the nanoparticles have an average diameter of from about 10 nm to about 150 nm after reconstitution. In some embodiments, the nanoparticles have an average diameter of from about 10 nm to about 140 nm after reconstitution. In some embodiments, the nanoparticles have an average diameter of from about 10 nm to about 130 nm after reconstitution. In some embodiments, the nanoparticles have an average diameter of from about 10 nm to about 120 nm after reconstitution. In some embodiments, the nanoparticles have an average diameter of from about 10 nm to about 110 nm after reconstitution. In some embodiments, the nanoparticles have an average diameter of from about 10 nm to about 100 nm after reconstitution. In some embodiments, the nanoparticles have an average diameter of from about 10 nm to about 90 nm after reconstitution. In some embodiments, the nanoparticles have an average diameter of from about 10 nm to about 80 nm after reconstitution. In some embodiments, the nanoparticles have an average diameter of from about 10 nm to about 70 nm after reconstitution. In some embodiments, the nanoparticles have an average diameter of from about 10 nm to about 60 nm after reconstitution. In some embodiments, the nanoparticles have an average diameter of from about 10 nm to about 50 nm after reconstitution. In some embodiments, the nanoparticles have an average diameter of from about 10 nm to about 40 nm after reconstitution. In some embodiments, the nanoparticles have an average diameter of from about 10 nm to about 30 nm after reconstitution. In some embodiments, the nanoparticles have an average diameter of from about 10 nm to about 20 nm after reconstitution.

In some embodiments, the nanoparticles have an average diameter of from about 20 nm to about 1000 nm after reconstitution. In some embodiments, the nanoparticles have an average diameter of from about 20 nm to about 950 nm after reconstitution. In some embodiments, the nanoparticles have an average diameter of from about 20 nm to about 900 nm after reconstitution. In some embodiments, the nanoparticles have an average diameter of from about 20 nm to about 850 nm after reconstitution. In some embodiments, the nanoparticles have an average diameter of from about 20 nm to about 800 nm after reconstitution. In some embodiments, the nanoparticles have an average diameter of from about 20 nm to about 750 nm after reconstitution. In some embodiments, the nanoparticles have an average diameter of from about 20 nm to about 700 nm after reconstitution. In some embodiments, the nanoparticles have an average diameter of from about 20 nm to about 650 nm after reconstitution. In some embodiments, the nanoparticles have an average diameter of from about 20 nm to about 600 nm after reconstitution. In some embodiments, the nanoparticles have an average diameter of from about 20 nm to about 550 nm after reconstitution. In some embodiments, the nanoparticles have an average diameter of from about 20 nm to about 500 nm after reconstitution. In some embodiments, the nanoparticles have an average diameter of from about 20 nm to about 450 nm after reconstitution. In some embodiments, the nanoparticles have an average diameter of from about 20 nm to about 400 nm after reconstitution. In some embodiments, the nanoparticles have an average diameter of from about 20 nm to about 350 nm after reconstitution. In some embodiments, the nanoparticles have an average diameter of from about 20 nm to about 300 nm after reconstitution. In some embodiments, the nanoparticles have an average diameter of from about 20 nm to about 250 nm after reconstitution. In some embodiments, the nanoparticles have an average diameter of from about 20 nm to about 240 nm after reconstitution. In some embodiments, the nanoparticles have an average diameter of from about 20 nm to about 230 nm after reconstitution. In some embodiments, the nanoparticles have an average diameter of from about 20 nm to about 220 nm after reconstitution. In some embodiments, the nanoparticles have an average diameter of from about 20 nm to about 210 nm after reconstitution. In some embodiments, the nanoparticles have an average diameter of from about 20 nm to about 200 nm after reconstitution. In some embodiments, the nanoparticles have an average diameter of from about 20 nm to about 190 nm after reconstitution. In some embodiments, the nanoparticles have an average diameter of from about 20 nm to about 180 nm after reconstitution. In some embodiments, the nanoparticles have an average diameter of from about 20 nm to about 170 nm after reconstitution. In some embodiments, the nanoparticles have an average diameter of from about 20 nm to about 160 nm after reconstitution. In some embodiments, the nanoparticles have an average diameter of from about 20 nm to about 150 nm after reconstitution. In some embodiments, the nanoparticles have an average diameter of from about 20 nm to about 140 nm after reconstitution. In some embodiments, the nanoparticles have an average diameter of from about 20 nm to about 130 nm after reconstitution. In some embodiments, the nanoparticles have an average diameter of from about 20 nm to about 120 nm after reconstitution. In some embodiments, the nanoparticles have an average diameter of from about 20 nm to about 110 nm after reconstitution. In some embodiments, the nanoparticles have an average diameter of from about 20 nm to about 100 nm after reconstitution. In some embodiments, the nanoparticles have an average diameter of from about 20 nm to about 90 nm after reconstitution. In some embodiments, the nanoparticles have an average diameter of from about 20 nm to about 80 nm after reconstitution. In some embodiments, the nanoparticles have an average diameter of from about 20 nm to about 70 nm after reconstitution. In some embodiments, the nanoparticles have an average diameter of from about 20 nm to about 60 nm after reconstitution. In some embodiments, the nanoparticles have an average diameter of from about 20 nm to about 50 nm after reconstitution. In some embodiments, the nanoparticles have an average diameter of from about 20 nm to about 40 nm after reconstitution. In some embodiments, the nanoparticles have an average diameter of from about 20 nm to about 30 nm after reconstitution.

In some embodiments, the nanoparticles have an average diameter of from about 30 nm to about 1000 nm after reconstitution. In some embodiments, the nanoparticles have an average diameter of from about 30 nm to about 950 nm after reconstitution. In some embodiments, the nanoparticles have an average diameter of from about 30 nm to about 900 nm after reconstitution. In some embodiments, the nanoparticles have an average diameter of from about 30 nm to about 850 nm after reconstitution. In some embodiments, the nanoparticles have an average diameter of from about 30 nm to about 800 nm after reconstitution. In some embodiments, the nanoparticles have an average diameter of from about 30 nm to about 750 nm after reconstitution. In some embodiments, the nanoparticles have an average diameter of from about 30 nm to about 700 nm after reconstitution. In some embodiments, the nanoparticles have an average diameter of from about 30 nm to about 650 nm after reconstitution. In some embodiments, the nanoparticles have an average diameter of from about 30 nm to about 600 nm after reconstitution. In some embodiments, the nanoparticles have an average diameter of from about 30 nm to about 550 nm after reconstitution. In some embodiments, the nanoparticles have an average diameter of from about 30 nm to about 500 nm. In some embodiments, the nanoparticles have an average diameter of from about 30 nm to about 450 nm after reconstitution. In some embodiments, the nanoparticles have an average diameter of from about 30 nm to about 400 nm after reconstitution. In some embodiments, the nanoparticles have an average diameter of from about 30 nm to about 350 nm after reconstitution. In some embodiments, the nanoparticles have an average diameter of from about 30 nm to about 300 nm after reconstitution. In some embodiments, the nanoparticles have an average diameter of from about 30 nm to about 250 nm after reconstitution. In some embodiments, the nanoparticles have an average diameter of from about 30 nm to about 240 nm after reconstitution. In some embodiments, the nanoparticles have an average diameter of from about 30 nm to about 230 nm after reconstitution. In some embodiments, the nanoparticles have an average diameter of from about 30 nm to about 220 nm after reconstitution. In some embodiments, the nanoparticles have an average diameter of from about 30 nm to about 210 nm after reconstitution. In some embodiments, the nanoparticles have an average diameter of from about 30 nm to about 200 nm after reconstitution. In some embodiments, the nanoparticles have an average diameter of from about 30 nm to about 190 nm after reconstitution. In some embodiments, the nanoparticles have an average diameter of from about 30 nm to about 180 nm after reconstitution. In some embodiments, the nanoparticles have an average diameter of from about 30 nm to about 170 nm after reconstitution. In some embodiments, the nanoparticles have an average diameter of from about 30 nm to about 160 nm after reconstitution. In some embodiments, the nanoparticles have an average diameter of from about 30 nm to about 150 nm after reconstitution. In some embodiments, the nanoparticles have an average diameter of from about 30 nm to about 140 nm after reconstitution. In some embodiments, the nanoparticles have an average diameter of from about 30 nm to about 130 nm after reconstitution. In some embodiments, the nanoparticles have an average diameter of from about 30 nm to about 120 nm after reconstitution. In some embodiments, the nanoparticles have an average diameter of from about 30 nm to about 110 nm after reconstitution. In some embodiments, the nanoparticles have an average diameter of from about 30 nm to about 100 nm after reconstitution. In some embodiments, the nanoparticles have an average diameter of from about 30 nm to about 90 nm after reconstitution. In some embodiments, the nanoparticles have an average diameter of from about 30 nm to about 80 nm after reconstitution. In some embodiments, the nanoparticles have an average diameter of from about 30 nm to about 70 nm after reconstitution. In some embodiments, the nanoparticles have an average diameter of from about 30 nm to about 60 nm after reconstitution. In some embodiments, the nanoparticles have an average diameter of from about 30 nm to about 50 nm after reconstitution. In some embodiments, the nanoparticles have an average diameter of from about 30 nm to about 40 nm after reconstitution.

In some embodiments, the nanoparticles have an average diameter of from about 40 nm to about 1000 nm after reconstitution. In some embodiments, the nanoparticles have an average diameter of from about 40 nm to about 950 nm after reconstitution. In some embodiments, the nanoparticles have an average diameter of from about 40 nm to about 900 nm after reconstitution. In some embodiments, the nanoparticles have an average diameter of from about 40 nm to about 850 nm after reconstitution. In some embodiments, the nanoparticles have an average diameter of from about 40 nm to about 800 nm after reconstitution. In some embodiments, the nanoparticles have an average diameter of from about 40 nm to about 750 nm after reconstitution. In some embodiments, the nanoparticles have an average diameter of from about 40 nm to about 700 nm after reconstitution. In some embodiments, the nanoparticles have an average diameter of from about 40 nm to about 650 nm after reconstitution. In some embodiments, the nanoparticles have an average diameter of from about 40 nm to about 600 nm after reconstitution. In some embodiments, the nanoparticles have an average diameter of from about 40 nm to about 550 nm after reconstitution. In some embodiments, the nanoparticles have an average diameter of from about 40 nm to about 500 nm after reconstitution. In some embodiments, the nanoparticles have an average diameter of from about 40 nm to about 450 nm after reconstitution. In some embodiments, the nanoparticles have an average diameter of from about 40 nm to about 400 nm after reconstitution. In some embodiments, the nanoparticles have an average diameter of from about 40 nm to about 350 nm after reconstitution. In some embodiments, the nanoparticles have an average diameter of from about 40 nm to about 300 nm. In some embodiments, the nanoparticles have an average diameter of from about 40 nm to about 250 nm after reconstitution. In some embodiments, the nanoparticles have an average diameter of from about 40 nm to about 240 nm after reconstitution. In some embodiments, the nanoparticles have an average diameter of from about 40 nm to about 230 nm after reconstitution. In some embodiments, the nanoparticles have an average diameter of from about 40 nm to about 220 nm after reconstitution. In some embodiments, the nanoparticles have an average diameter of from about 40 nm to about 210 nm after reconstitution. In some embodiments, the nanoparticles have an average diameter of from about 40 nm to about 200 nm after reconstitution. In some embodiments, the nanoparticles have an average diameter of from about 40 nm to about 190 nm after reconstitution. In some embodiments, the nanoparticles have an average diameter of from about 40 nm to about 180 nm after reconstitution. In some embodiments, the nanoparticles have an average diameter of from about 40 nm to about 170 nm after reconstitution. In some embodiments, the nanoparticles have an average diameter of from about 40 nm to about 160 nm after reconstitution. In some embodiments, the nanoparticles have an average diameter of from about 40 nm to about 150 nm after reconstitution. In some embodiments, the nanoparticles have an average diameter of from about 40 nm to about 140 nm after reconstitution. In some embodiments, the nanoparticles have an average diameter of from about 40 nm to about 130 nm after reconstitution. In some embodiments, the nanoparticles have an average diameter of from about 40 nm to about 120 nm after reconstitution. In some embodiments, the nanoparticles have an average diameter of from about 40 nm to about 110 nm after reconstitution. In some embodiments, the nanoparticles have an average diameter of from about 40 nm to about 100 nm after reconstitution. In some embodiments, the nanoparticles have an average diameter of from about 40 nm to about 90 nm after reconstitution. In some embodiments, the nanoparticles have an average diameter of from about 40 nm to about 80 nm after reconstitution. In some embodiments, the nanoparticles have an average diameter of from about 40 nm to about 70 nm after reconstitution. In some embodiments, the nanoparticles have an average diameter of from about 40 nm to about 60 nm after reconstitution. In some embodiments, the nanoparticles have an average diameter of from about 40 nm to about 50 nm after reconstitution.

In some embodiments, the nanoparticles have an average diameter of from about 50 nm to about 1000 nm after reconstitution. In some embodiments, the nanoparticles have an average diameter of from about 50 nm to about 950 nm after reconstitution. In some embodiments, the nanoparticles have an average diameter of from about 50 nm to about 900 nm after reconstitution. In some embodiments, the nanoparticles have an average diameter of from about 50 nm to about 850 nm after reconstitution. In some embodiments, the nanoparticles have an average diameter of from about 50 nm to about 800 nm after reconstitution. In some embodiments, the nanoparticles have an average diameter of from about 50 nm to about 750 nm after reconstitution. In some embodiments, the nanoparticles have an average diameter of from about 50 nm to about 700 nm after reconstitution. In some embodiments, the nanoparticles have an average diameter of from about 50 nm to about 650 nm after reconstitution. In some embodiments, the nanoparticles have an average diameter of from about 50 nm to about 600 nm after reconstitution. In some embodiments, the nanoparticles have an average diameter of from about 50 nm to about 550 nm after reconstitution. In some embodiments, the nanoparticles have an average diameter of from about 50 nm to about 500 nm after reconstitution. In some embodiments, the nanoparticles have an average diameter of from about 50 nm to about 450 nm after reconstitution. In some embodiments, the nanoparticles have an average diameter of from about 50 nm to about 400 nm after reconstitution. In some embodiments, the nanoparticles have an average diameter of from about 50 nm to about 350 nm after reconstitution. In some embodiments, the nanoparticles have an average diameter of from about 50 nm to about 300 nm after reconstitution. In some embodiments, the nanoparticles have an average diameter of from about 50 nm to about 250 nm after reconstitution. In some embodiments, the nanoparticles have an average diameter of from about 50 nm to about 240 nm after reconstitution. In some embodiments, the nanoparticles have an average diameter of from about 50 nm to about 230 nm after reconstitution. In some embodiments, the nanoparticles have an average diameter of from about 50 nm to about 220 nm after reconstitution. In some embodiments, the nanoparticles have an average diameter of from about 50 nm to about 210 nm after reconstitution. In some embodiments, the nanoparticles have an average diameter of from about 50 nm to about 200 nm after reconstitution. In some embodiments, the nanoparticles have an average diameter of from about 50 nm to about 190 nm after reconstitution. In some embodiments, the nanoparticles have an average diameter of from about 50 nm to about 180 nm after reconstitution. In some embodiments, the nanoparticles have an average diameter of from about 50 nm to about 170 nm after reconstitution. In some embodiments, the nanoparticles have an average diameter of from about 50 nm to about 160 nm after reconstitution. In some embodiments, the nanoparticles have an average diameter of from about 50 nm to about 150 nm after reconstitution. In some embodiments, the nanoparticles have an average diameter of from about 50 nm to about 140 nm after reconstitution. In some embodiments, the nanoparticles have an average diameter of from about 50 nm to about 130 nm after reconstitution. In some embodiments, the nanoparticles have an average diameter of from about 50 nm to about 120 nm after reconstitution. In some embodiments, the nanoparticles have an average diameter of from about 50 nm to about 110 nm after reconstitution. In some embodiments, the nanoparticles have an average diameter of from about 50 nm to about 100 nm after reconstitution. In some embodiments, the nanoparticles have an average diameter of from about 50 nm to about 90 nm after reconstitution. In some embodiments, the nanoparticles have an average diameter of from about 50 nm to about 80 nm after reconstitution. In some embodiments, the nanoparticles have an average diameter of from about 50 nm to about 70 nm after reconstitution. In some embodiments, the nanoparticles have an average diameter of from about 50 nm to about 60 nm after reconstitution.

In some embodiments, the nanoparticles have an average diameter of about 10 nm after reconstitution. In some embodiments, the nanoparticles have an average diameter of about 20 nm after reconstitution. In some embodiments, the nanoparticles have an average diameter of about 30 nm after reconstitution. In some embodiments, the nanoparticles have an average diameter of about 40 nm after reconstitution. In some embodiments, the nanoparticles have an average diameter of about 50 nm after reconstitution. In some embodiments, the nanoparticles have an average diameter of about 60 nm after reconstitution. In some embodiments, the nanoparticles have an average diameter of about 70 nm after reconstitution. In some embodiments, the nanoparticles have an average diameter of about 80 nm after reconstitution. In some embodiments, the nanoparticles have an average diameter of about 90 nm after reconstitution. In some embodiments, the nanoparticles have an average diameter of about 100 nm after reconstitution. In some embodiments, the nanoparticles have an average diameter of about 110 nm after reconstitution. In some embodiments, the nanoparticles have an average diameter of about 120 nm after reconstitution. In some embodiments, the nanoparticles have an average diameter of about 130 nm after reconstitution. In some embodiments, the nanoparticles have an average diameter of about 140 nm after reconstitution. In some embodiments, the nanoparticles have an average diameter of about 150 nm after reconstitution. In some embodiments, the nanoparticles have an average diameter of about 160 nm after reconstitution. In some embodiments, the nanoparticles have an average diameter of about 170 nm after reconstitution. In some embodiments, the nanoparticles have an average diameter of about 180 nm. In some embodiments, the nanoparticles have an average diameter of about 190 nm after reconstitution. In some embodiments, the nanoparticles have an average diameter of about 200 nm after reconstitution. In some embodiments, the nanoparticles have an average diameter of about 210 nm after reconstitution. In some embodiments, the nanoparticles have an average diameter of about 220 nm after reconstitution. In some embodiments, the nanoparticles have an average diameter of about 230 nm after reconstitution. In some embodiments, the nanoparticles have an average diameter of about 240 nm after reconstitution. In some embodiments, the nanoparticles have an average diameter of about 250 nm after reconstitution. In some embodiments, the nanoparticles have an average diameter of about 300 nm after reconstitution. In some embodiments, the nanoparticles have an average diameter of about 350 nm after reconstitution. In some embodiments, the nanoparticles have an average diameter of about 400 nm after reconstitution. In some embodiments, the nanoparticles have an average diameter of about 450 nm after reconstitution. In some embodiments, the nanoparticles have an average diameter of about 500 nm after reconstitution. In some embodiments, the nanoparticles have an average diameter of about 550 nm after reconstitution. In some embodiments, the nanoparticles have an average diameter of about 600 nm after reconstitution. In some embodiments, the nanoparticles have an average diameter of about 650 nm after reconstitution. In some embodiments, the nanoparticles have an average diameter of about 700 nm after reconstitution. In some embodiments, the nanoparticles have an average diameter of about 750 nm after reconstitution. In some embodiments, the nanoparticles have an average diameter of about 800 nm. In some embodiments, the nanoparticles have an average diameter of about 850 nm after reconstitution. In some embodiments, the nanoparticles have an average diameter of about 900 nm after reconstitution. In some embodiments, the nanoparticles have an average diameter of about 950 nm after reconstitution. In some embodiments, the nanoparticles have an average diameter of about 1000 nm after reconstitution.

### Preparation of Nanoparticles

Provided in another aspect is a process of preparing a nanoparticle composition comprising:
a) dissolving a compound of Formula (I), or a pharmaceutically acceptable salt thereof, in a volatile solvent to form a solution comprising a dissolved compound of Formula (I), or a pharmaceutically acceptable salt thereof;
b) adding the solution comprising the dissolved compound of Formula (I), or a pharmaceutically acceptable salt thereof, to a pharmaceutically acceptable carrier in an aqueous solution to form an emulsion;
c) subjecting the emulsion to homogenization to form a homogenized emulsion; and
d) subjecting the homogenized emulsion to evaporation of the volatile solvent to form the nanoparticle composition;
   wherein the nanoparticles comprise a compound of Formula (I), or a pharmaceutically acceptable salt thereof, and a pharmaceutically acceptable carrier, wherein the pharmaceutically acceptable carrier comprises albumin and the compound of Formula (I) has the structure:

A-L-B Formula (I);

wherein:
A is a compound that binds to an E3 ubiquitin ligase;
L is a linker comprising at least two carbon atoms; and
B is a ligand which binds to a target protein or polypeptide which is to be mono-ubiquitinated or poly-ubiquitinated by the E3 ligase and thereby degraded, and is linked to the A group through the L group.

In some embodiments, the adding the solution comprising the dissolved compound of Formula (I), or a pharmaceutically acceptable salt thereof, to a pharmaceutically acceptable carrier in an aqueous solution from step b) further comprises mixing to form an emulsion. In some embodiments, the mixing is performed with a homogenizer. In some embodiments, the volatile solvent is a chlorinated solvent, alcohol, ketone, ester, ether, acetonitrile, or any combination thereof. In some embodiments, volatile solvent is a chlorinated solvent. Examples of chlorinated solvents include, but are not limited to, chloroform, dichloromethane, and 1,2, dichloroethane. In some embodiments, volatile solvent is an alcohol. Examples of alcohols, include but are not limited to, methanol, ethanol, butanol (such as t-butyl and n-butyl alcohol), and propanol (such as iso-propyl alcohol). In some embodiments, volatile solvent is a ketone. An example of a ketone includes, but is not limited to, acetone. In some embodiments, volatile solvent is an ester. An example of an ester includes, but is not limited to ethyl acetate. In some embodiments, volatile solvent is an ether. In some embodiments, the volatile solvent is acetonitrile. In some embodiments, the volatile solvent is mixture of a chlorinated solvent with an alcohol.

In some embodiments, the volatile solvent is chloroform, ethanol, butanol, methanol, propanol, or a combination thereof. In some embodiments, volatile solvent is a mixture of chloroform and ethanol. In some embodiments, the volatile solvent is methanol. In some embodiments, the volatile solvent is a mixture of chloroform and methanol. In some embodiments, the volatile solvent is butanol, such as t-butanol or n-butanol. In some embodiments, the volatile solvent is a mixture of chloroform and butanol. In some embodiments, the volatile solvent is acetone. In some embodiments, the volatile solvent is acetonitrile. In some embodiments, the volatile solvent is dichloromethane. In some embodiments, the volatile solvent is 1,2 dichloroethane. In some embodiments the volatile solvent is ethyl acetate. In some embodiments, the volatile solvent is isopropyl alcohol. In some embodiments, the volatile solvent is chloroform. In some embodiments, the volatile solvent is ethanol. In some embodiments, the volatile solvent is a combination of ethanol and chloroform.

In some embodiments, the homogenization is high pressure homogenization. In some embodiments, the emulsion is cycled through high pressure homogenization for an appropriate amount of cycles. In some embodiments, the appropriate amount of cycles is from about 2 to about 10 cycles. In some embodiments, the appropriate amount of cycles is about 1, about 2, about 3, about 4, about 5, about 6, about 7, about 8, about 9, or about 10 cycles.

In some embodiments, the evaporation is accomplished with suitable equipment known for this purpose. Such suitable equipment include, but not limited to, rotary evaporators, falling film evaporators, wiped film evaporators, spray driers, and the like that can be operated in batch mode or in continuous operation. In some embodiments, the evaporation is accomplished with a rotary evaporator. In some embodiments, the evaporation is under reduced pressure.

### Administration

In some embodiments, the composition is suitable for injection. In some embodiments, the composition is suitable for parenteral administration. Examples of parenteral administration include but are not limited to subcutaneous injections, intravenous, or intramuscular injections or infusion techniques. In some embodiments, the composition is suitable for intravenous administration.

In some embodiments, the composition is administered intraperitoneally, intraarterially, intrapulmonarily, orally, by inhalation, intravesicularly, intramuscularly, intratracheally, subcutaneously, intraocularly, intrathecally, intratumorally, or transdermally. In some embodiments, the composition is administered intravenously. In some embodiments, the composition is administered intraarterially. In some embodiments, the composition is administered intrapulmonarily. In some embodiments, the composition is administered orally. In some embodiments, the composition is administered by inhalation. In some embodiments, the composition is administered intravesicularly. In some embodiments, the composition is administered intramuscularly. In some embodiments, the composition is administered intratracheally. In some embodiments, the composition is administered subcutaneously. In some embodiments, the composition is administered intraocularly. In some embodiments, the composition is administered intrathecally. In some embodiments, the composition is administered transdermally.

### Methods

Also provided herein in another aspect is a method of treating a disease in a subject in need thereof comprising administering any one of the compositions described herein.

Also disclosed herein is a method of delivering a compound of Formula (I), or a pharmaceutically acceptable salt thereof, to a subject in need thereof comprising administering any one of the compositions described herein.

Disclosed compositions are administered to patients (animals and humans) in need of such treatment in dosages that will provide optimal pharmaceutical efficacy. It will be appreciated that the dose required for use in any particular application will vary from patient to patient, not only with the particular composition selected, but also with the route of administration, the nature of the condition being treated, the age and condition of the patient, concurrent medication or special diets then being followed by the patient, and other factors, with the appropriate dosage ultimately being at the discretion of the attendant physician. In some embodiments, a contemplated composition disclosed herein is administered orally, subcutaneously, topically, parenterally, by inhalation spray, or rectally in dosage unit formulations containing conventional non-toxic pharmaceutically acceptable carriers, adjuvants and vehicles. Parenteral administration include subcutaneous injections, intravenous, or intramuscular injections or infusion techniques.

The following examples are provided merely as illustrative of various embodiments and shall not be construed to limit the invention in any way.

### EXAMPLES

### Exemplary Nanoparticle Compositions Containing Heterobifunctional Molecules for Specific Target Degradation

### Example 1: Nanoparticle Pharmaceutical Composition Comprising Compound 1 (A=Cereblon Binder; B=BRD4 Binder) and Albumin

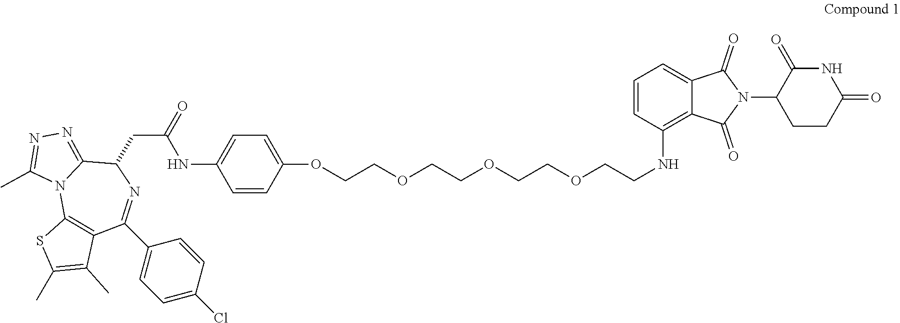 [SMILES: Cc1sc2c(c1C)C(c1ccc(Cl)cc1)=N[C@@H](CC(=O)Nc1ccc(OCCOCCOCCOCCNc3cccc4c3C(=O)N(C3CCC(=O)NC3=O)C4=O)cc1)c1nnc(C)n1-2]

14.7 mL of a human albumin solution (1.47% w/v) was prepared diluting from a 25% human albumin U.S.P. solution using chloroform saturated water. Compound 1 (24 mg) was dissolved in 300 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-05), where emulsification was performed by recycling the emulsion for 2 minutes at high pressure (12,000 psi to 20,000 psi) while cooling (4° to 8° C.). The resulting emulsion was transferred into a rotary evaporator (Buchi, Switzerland), where the volatile solvents were removed at 40° C. under reduced pressure (approximately 25 mm Hg) for 4 minutes. The suspension was then sterile filtered, and the average particle size (Zₐᵥ, Malvern Nano-S) was determined to be 105 nm initially, 104 nm after 30 minutes, 105 nm after 60 minutes, 106 nm after 120 minutes, 106 nm after 44 hours, and 108 nm after 9 days at room temperature.

### Example 2: Nanoparticle Pharmaceutical Composition Comprising Compound 2 (A=Cereblon Binder; B=BET Binder) and Albumin

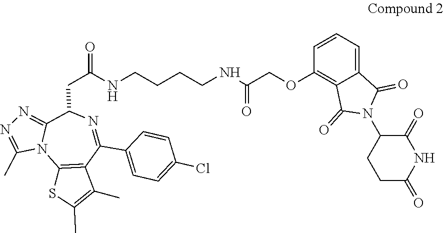 [SMILES: Cc1sc2c(c1C)C(c1ccc(Cl)cc1)=N[C@@H](CC(=O)NCCCCNC(=O)COc1cccc3c1C(=O)N(C1CCC(=O)NC1=O)C3=O)c1nnc(C)n1-2]

29.4 mL of a human albumin solution (1.47% w/v) was prepared diluting from a 25% human albumin U.S.P. solution using chloroform saturated water. Compound 2 (40 mg) was dissolved in 600 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-05), where emulsification was performed by recycling the emulsion for 2 minutes at high pressure (12,000 psi to 20,000 psi) while cooling (4° to 8° C.). The resulting emulsion was transferred into a rotary evaporator (Buchi, Switzerland), where the volatile solvents were removed at 40° C. under reduced pressure (approximately 25 mm Hg) for 7 minutes. The suspension was then filtered at 0.45 μm, and the average particle size (Zₐᵥ, Malvern Nano-S) was determined to be 163 nm initially, 160 nm after 30 minutes, 162 nm after 120 minutes, 164 nm after 240 minutes, 173 nm after 28 hours at room temperature.

### Example 3: Nanoparticle Pharmaceutical Composition Comprising Compound 3 (A=VHL Binder; B=BET Binder) and Albumin

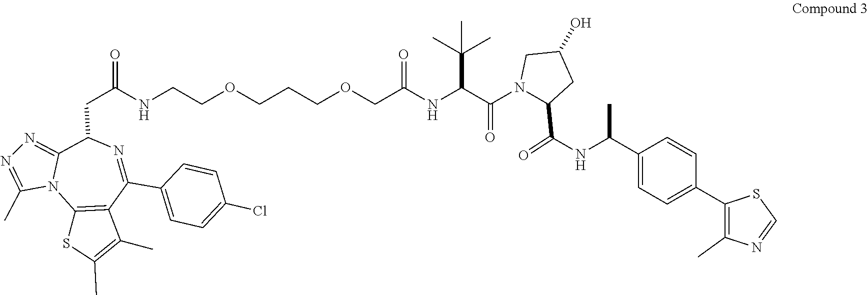 [SMILES: Cc1ncsc1-c1ccc([C@H](C)NC(=O)[C@@H]2C[C@@H](O)CN2C(=O)[C@@H](NC(=O)COCCCOCCNC(=O)C[C@@H]2N=C(c3ccc(Cl)cc3)c3c(sc(C)c3C)-n3c(C)nnc32)C(C)(C)C)cc1]

14.7 mL of a human albumin solution (1.47% w/v) was prepared diluting from a 25% human albumin U.S.P. solution using chloroform saturated water. Compound 3 (24 mg) was dissolved in 225 μL chloroform/ethanol (80:20 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-05), where emulsification was performed by recycling the emulsion for 2 minutes at high pressure (12,000 psi to 20,000 psi) while cooling (4° to 8° C.). The resulting emulsion was transferred into a rotary evaporator (Buchi, Switzerland), where the volatile solvents were removed at 40° C. under reduced pressure (approximately 25 mm Hg) for 6 minutes. The suspension was then filtered at 0.8 μm, and the average particle size (Zₐᵥ, Malvern Nano-S) was determined to be 269 nm initially, 342 nm after 15 minutes, 360 nm after 30 minutes, 385 nm after 60 minutes, and 417 nm after 120 minutes at room temperature. By 18 hrs at room temperature, the particles were unstable had aggregated into multiple distinct particle sizes.

### Example 4: Nanoparticle Pharmaceutical Composition Comprising Compound 3 (A=Cereblon Binder; B=CDK9 Binder) and Albumin

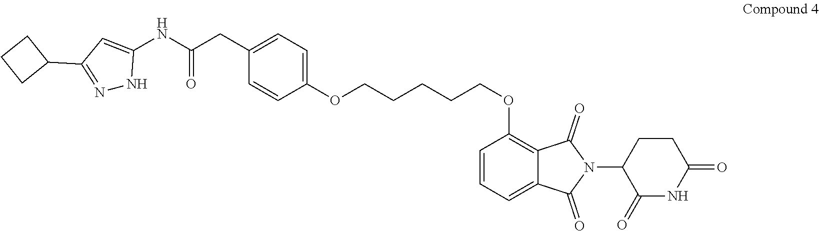 [SMILES: O=C1CCC(N2C(=O)c3cccc(OCCCCCOc4ccc(CC(=O)Nc5cc(C6CCC6)n[nH]5)cc4)c3C2=O)C(=O)N1]

19.6 mL of a human albumin solution (1.47% w/v) was prepared diluting from a 25% human albumin U.S.P. solution using chloroform saturated water. Compound 3 (21 mg) was dissolved in 440 μL chloroform/ethanol (90:10 ratio). The organic solvent solution was added drop wise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-05), where emulsification was performed by recycling the emulsion for 2 minutes at high pressure (12,000 psi to 20,000 psi) while cooling (4° to 8° C.). The resulting emulsion was transferred into a rotary evaporator (Buchi, Switzerland), where the volatile solvents were removed at 40° C. under reduced pressure (approximately 25 mm Hg) for 6 minutes. The suspension was then sterile filtered, and the average particle size (Zₐᵥ, Malvern Nano-S) was determined to be 90 nm initially, 90 nm after 30 minutes, 90 nm after 80 minutes, 90 nm after 120 minutes, 88 nm after 4 hours, and 90 nm after 24 hours at room temperature.

### Example 5: Nanoparticle Pharmaceutical Composition Comprising Compound 5 (A=MDM2 Binder; B=BRD4 Binder) and Albumin

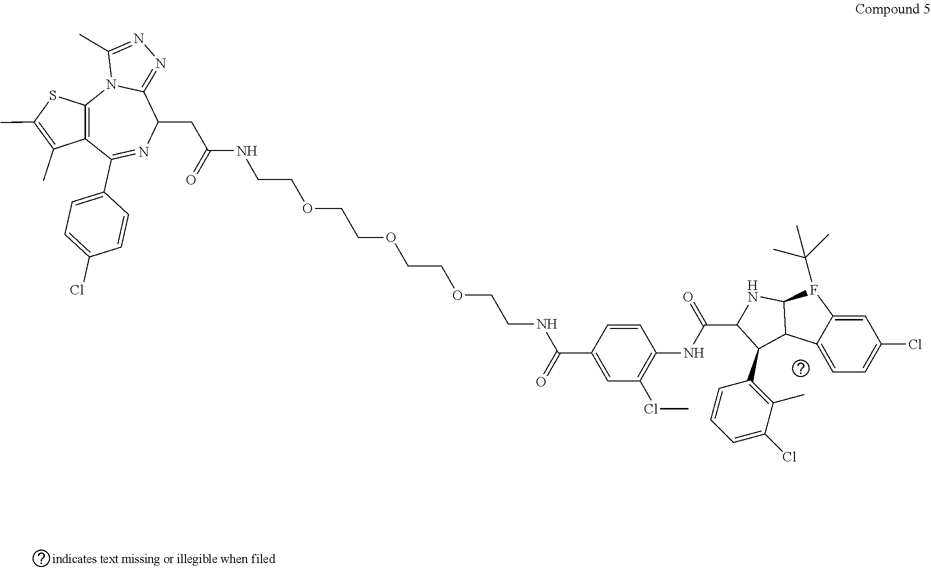

19.6 mL of a human albumin solution (1.47% w/v) was prepared diluting from a 25% human albumin U.S.P. solution using chloroform saturated water. Compound 5 (40 mg) was dissolved in 400 μL chloroform/ethanol (90:10). The organic solvent solution was added drop wise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-05), where emulsification was performed by recycling the emulsion for 2 minutes at high pressure (12,000 psi to 20,000 psi) while cooling (4° to 8° C.). The resulting emulsion was transferred into a rotary evaporator (Buchi, Switzerland), where the volatile solvents were removed at 40° C. under reduced pressure (approximately 25 mm Hg) for 5 minutes. The suspension was then sterile filtered, and the average particle size (Zₐᵥ, Malvern Nano-S) was determined to be 92 nm initially, 91 nm after 60 minutes, 91 nm after 4 hours, and 93 nm after 26 hours at room temperature.

### Example 6: Nanoparticle Pharmaceutical Composition Comprising Compound 6 (A=VHL Binder; B=BRD4 Binder) and Albumin

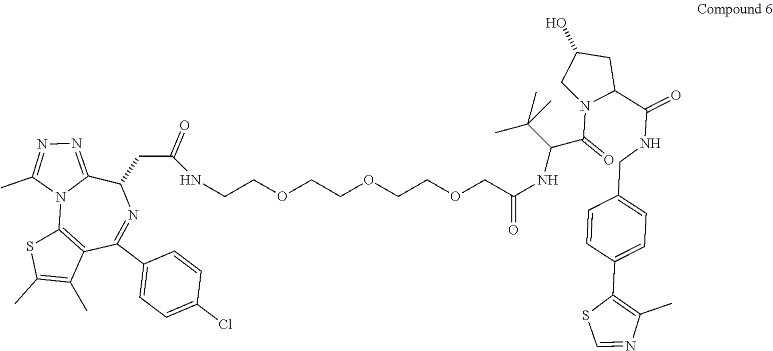 [SMILES: Cc1ncsc1-c1ccc(CNC(=O)C2C[C@@H](O)CN2C(=O)C(NC(=O)COCCOCCOCCNC(=O)C[C@@H]2N=C(c3ccc(Cl)cc3)c3c(sc(C)c3C)-n3c(C)nnc32)C(C)(C)C)cc1]

19.6 mL of a human albumin solution (1.47% w/v) was prepared diluting from a 25% human albumin U.S.P. solution using chloroform saturated water. Compound 6 (34 mg) was dissolved in 400 μL chloroform/ethanol (90:10). The organic solvent solution was added drop wise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-05), where emulsification was performed by recycling the emulsion for 2 minutes at high pressure (12,000 psi to 20,000 psi) while cooling (4° to 8° C.). The resulting emulsion was transferred into a rotary evaporator (Buchi, Switzerland), where the volatile solvents were removed at 40° C. under reduced pressure (approximately 25 mm Hg) for 5 minutes. The suspension was then filtered at 0.8 μm, and the average particle size (Zₐᵥ, Malvern Nano-S) was determined to be 204 nm initially, 238 nm after 15 minutes, 250 nm after 30 minutes, 273 nm after 60 minutes, 315 nm after 2 hours, and 400 nm after 24 hours at room temperature.

### Example 7: Nanoparticle Pharmaceutical Composition Comprising Compound 7 (A=VHL Binder; B=BRD4 Binder) and Albumin

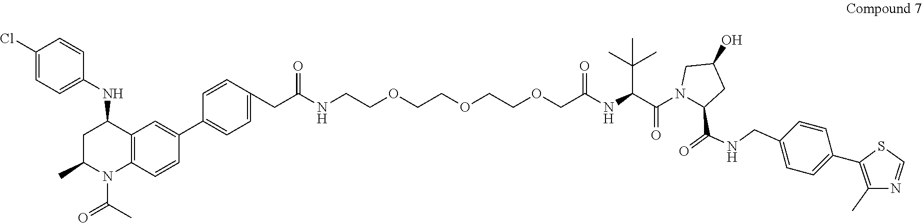 [SMILES: CC(=O)N1c2ccc(-c3ccc(CC(=O)NCCOCCOCCOCC(=O)N[C@H](C(=O)N4C[C@@H](O)C[C@H]4C(=O)NCc4ccc(-c5scnc5C)cc4)C(C)(C)C)cc3)cc2[C@H](Nc2ccc(Cl)cc2)C[C@@H]1C]

19.6 mL of a human albumin solution (1.47% w/v) was prepared diluting from a 25% human albumin U.S.P. solution using chloroform saturated water. Compound 7 (36 mg) was dissolved in 400 μL chloroform/ethanol (90:10). The organic solvent solution was added drop wise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-05), where emulsification was performed by recycling the emulsion for 2 minutes at high pressure (12,000 psi to 20,000 psi) while cooling (4° to 8° C.). The resulting emulsion was transferred into a rotary evaporator (Buchi, Switzerland), where the volatile solvents were removed at 40° C. under reduced pressure (approximately 25 mm Hg) for 5 minutes. The suspension was then filtered at 0.8 μm, and the average particle size (Zₐᵥ, Malvern Nano-S) was determined to be 172 nm initially, 193 nm after 30 minutes, 202 nm after 60 minutes, 212 nm after 2 hours, and 244 nm after 24 hours at room temperature.

### Exemplary Nanoparticle Compositions Upon Lyophilization and Rehydration

### Example 8

This example demonstrates the lyophilization and rehydration into each of: water, 5% dextrose water, and saline for a nanoparticle pharmaceutical composition comprising Compound 1 and albumin. Immediately after sterile filtration, the nanoparticle suspension from Example 1 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 106 nm initially, 107 nm after 60 minutes, 106 nm after 2 hours, and 108 nm after 24 hrs at room temperature. Upon hydration into 5% dextrose water, the average particle size (Zₐᵥ, Malvern Nano-S) was determined to be 119 nm initially, 119 nm after 60 minutes, 118 nm after 2 hours, and 123 nm after 24 hrs at room temperature. Upon hydration into 0.9% saline, the average particle size (Zₐᵥ, Malvern Nano-S) was determined to be 107 nm initially, 106 nm after 60 minutes, 106 nm after 2 hours, and 106 nm after 24 hrs at room temperature.

### Example 9

This example demonstrates the lyophilization and rehydration into each of: water, 5% dextrose water, and saline for a nanoparticle pharmaceutical composition comprising Compound 2 and albumin. Immediately after 0.45 μm filtration, the nanoparticle suspension from Example 2 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 179 nm initially, 178 nm after 60 minutes, 185 nm after 2 hours, and 176 nm after 24 hrs at room temperature. Upon hydration into 5% dextrose water, the average particle size (Zₐᵥ, Malvern Nano-S) was determined to be 201 nm initially, 198 nm after 60 minutes, 196 nm after 2 hours, and 199 nm after 24 hrs at room temperature. Upon hydration into 0.9% saline, the average particle size (Zₐᵥ, Malvern Nano-S) was determined to be 185 nm initially, 190 nm after 60 minutes, 191 nm after 2 hours, and 210 nm after 24 hrs at room temperature.

### Example 10

This example demonstrates the lyophilization and rehydration into each of: water, 5% dextrose water, and saline for a nanoparticle pharmaceutical composition comprising Compound 3 and albumin. Immediately after 0.8 μm filtration, the nanoparticle suspension from Example 3 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 339 nm initially, 353 nm after 60 minutes, and 390 nm after 2 hours at room temperature. Upon hydration into 5% dextrose water, the average particle size (Zₐᵥ, Malvern Nano-S) was determined to be 287 nm initially, 429 nm after 60 minutes, and 462 nm after 2 hours at room temperature. Upon hydration into 0.9% saline, the average particle size (Zₐᵥ, Malvern Nano-S) was determined to be 236 nm initially, 337 nm after 60 minutes, and 384 nm after 2 hours at room temperature.

### Example 11

This example demonstrates the lyophilization and rehydration into each of: water, 5% dextrose water, and saline for a nanoparticle pharmaceutical composition comprising Compound 4 and albumin. Immediately after sterile filtration, the nanoparticle suspension from Example 4 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 91 nm initially, 90 nm after 60 minutes, 89 nm after 2 hours, and 89 nm after 24 hrs at room temperature. Upon hydration into 5% dextrose water, the average particle size (Zₐᵥ, Malvern Nano-S) was determined to be 101 nm initially, 101 nm after 60 minutes, 101 nm after 2 hours, and 100 nm after 24 hrs at room temperature. Upon hydration into 0.9% saline, the average particle size (Zₐᵥ, Malvern Nano-S) was determined to be 88 nm initially, 89 nm after 60 minutes, and 89 nm after 2 hours, and 89 nm after 24 hrs at room temperature.

### Example 12

This example demonstrates the lyophilization and rehydration into each of: water, 5% dextrose water, and saline for a nanoparticle pharmaceutical composition comprising Compound 5 and albumin. Immediately after sterile filtration, the nanoparticle suspension from Example 5 was flash frozen in liquid nitrogen, followed by complete lyophilization overnight to yield a dry cake, and stored at −20° C. The cake was then reconstituted. Upon hydration into water, the average particle size (Zₐᵥ, Malvern Nano-S) was determined to be 92 nm initially, 92 nm after 60 minutes, 92 nm after 2 hours, and 89 nm after 26 hours at room temperature. Upon hydration into 5% dextrose water, the average particle size (Zₐᵥ, Malvern Nano-S) was determined to be 107 nm initially, 107 nm after 60 minutes, 107 nm after 2 hours, and 107 nm after 26 hours at room temperature. Upon hydration into 0.9% saline, the average particle size (Zₐᵥ, Malvern Nano-S) was determined to be 91 nm initially, 91 nm after 60 minutes, and 91 nm after 2 hours, and 93 nm after 26 hours at room temperature.

### Example 13

This example demonstrates the lyophilization and rehydration into each of: water, 5% dextrose water, and saline for a nanoparticle pharmaceutical composition comprising Compound 6 and albumin. Immediately after 0.8 μm filtration, the nanoparticle suspension from Example 6 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 256 nm initially, 274 nm after 60 minutes, and 289 nm after 2 hours, and 380 nm after 26 hours at room temperature. Upon hydration into 5% dextrose water, the average particle size (Zₐᵥ, Malvern Nano-S) was determined to be 299 nm initially, 336 nm after 60 minutes, 355 nm after 2 hours, and 454 nm after 26 hours at room temperature. Upon hydration into 0.9% saline, the average particle size (Zₐᵥ, Malvern Nano-S) was determined to be 272 nm initially, 283 nm after 60 minutes, and 320 nm after 2 hours, and 366 nm after 26 hours at room temperature.

### Example 14

This example demonstrates the lyophilization and rehydration into each of: water, 5% dextrose water, and saline for a nanoparticle pharmaceutical composition comprising Compound 7 and albumin. Immediately after 0.8 μm filtration, the nanoparticle suspension from Example 7 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 223 nm initially, 240 nm after 60 minutes, 238 nm after 2 hours, and 302 nm after 26 hours at room temperature. Upon hydration into 5% dextrose water, the average particle size (Zₐᵥ, Malvern Nano-S) was determined to be 249 nm initially, 257 nm after 60 minutes, 275 nm after 2 hours, and 332 nm after 26 hours at room temperature. Upon hydration into 0.9% saline, the average particle size (Zₐᵥ, Malvern Nano-S) was determined to be 230 nm initially, 245 nm after 60 minutes, and 263 nm after 2 hours, and 298 nm after 26 hours at room temperature.

### Example of No Albumin Nanoparticles Produced when Using Some VHL-Containing Heterobifunctional Compounds

### Example 15: Compound 8 (A=VHL Binder; B=BRD4 Binder)

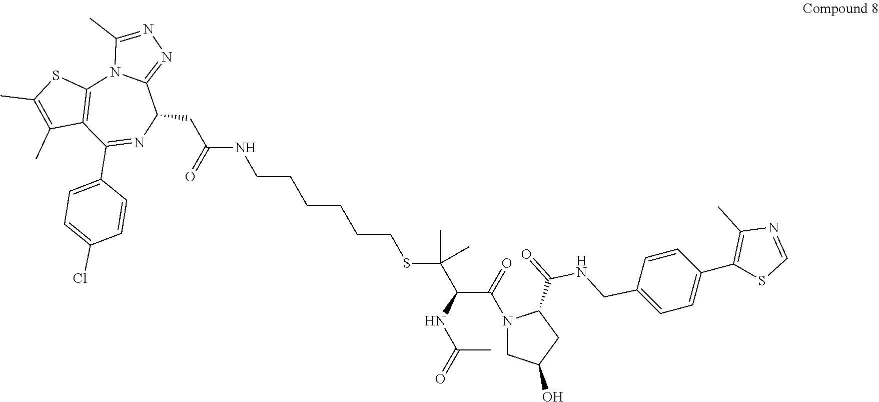 [SMILES: CC(=O)N[C@H](C(=O)N1C[C@H](O)C[C@H]1C(=O)NCc1ccc(-c2scnc2C)cc1)C(C)(C)SCCCCCCNC(=O)C[C@@H]1N=C(c2ccc(Cl)cc2)c2c(sc(C)c2C)-n2c(C)nnc21]

14.7 mL of a human albumin solution (1.47% w/v) was prepared diluting from a 25% human albumin U.S.P. solution using chloroform saturated water. Compound 8 (25 mg) was dissolved in 300 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-05), where emulsification was performed by recycling the emulsion for 2 minutes at high pressure (12,000 psi to 20,000 psi) while cooling (4° to 8° C.). The resulting emulsion was transferred into a rotary evaporator (Buchi, Switzerland), where the volatile solvents were removed at 40° C. under reduced pressure (approximately 25 mm Hg) for 5 minutes. The resulting solution was then filtered at 0.45 μm, and the average particle size (Zₐᵥ, Malvern Nano-S) was determined to be <15 nm, denoting only free albumin without nanoparticle formation.

What is claimed is:

1. A composition comprising nanoparticles, wherein the nanoparticles comprise a compound of Formula (I), or a pharmaceutically acceptable salt thereof, and a pharmaceutically acceptable carrier; wherein the pharmaceutically acceptable carrier comprises albumin and the compound of Formula (I) has the structure:
A-L-B Formula (I);
wherein:
A is a compound that binds to an E3 ubiquitin ligase;
L is a linker comprising at least two carbon atoms; and
B is a ligand which binds to a target protein or polypeptide which is to be mono-ubiquitinated or poly-ubiquitinated by the E3 ligase and thereby degraded, and is linked to the A group through the L group.

2. The composition of claim 1, wherein A is selected from a cereblon binder, a Von Hippel-Lindau tumor suppressor protein (VHL) binder, an inhibitor of apoptosis protein (IAP) binder, a Kelch-like ECH-associated protein 1 (Keap1) binder, a mouse double minute 2 homolog (MDM2) binder, and beta-transducin repeat containing protein (b-TrCP) binder.

3. The composition of claim 1 or 2, wherein A is a cereblon binder.

4. The composition of claim 3, wherein A is a cereblon binder selected from lenalidomide, pomalidomide, and thalidomide.

5. The composition of claim 1 or 2, wherein A is a VHL binder.

6. The composition of claim 1 or 2, wherein A is an IAP binder.

7. The composition of claim 3, wherein A is an IAP binder selected from an X-linked inhibitor of apoptosis protein (XIAP), cellular inhibitor of apoptosis protein-1 (cIAP1), cellular inhibitor of apoptosis protein-2 (cIAP2), neuronal apoptosis inhibitory protein (NAIP), livin, and survivin.

8. The composition of claim 1 or 2, wherein A is a Keap1 binder.

9. The composition of claim 1 or 2, wherein A is an MDM2 binder.

10. The composition of claim 1 or 2, wherein A is a b-TrCP binder.

11. The composition of any one of claims 1-10, wherein the nanoparticles have an average diameter of about 1000 nm or less for at least about 15 minutes after nanoparticle formation.

12. The composition of any one of claims 1-10, wherein the nanoparticles have an average diameter of about 10 nm or greater for at least about 15 minutes after nanoparticle formation.

13. The composition of any one of claims 1-10, the nanoparticles have an average diameter of from about 10 nm to about 1000 nm for at least about 15 minutes after nanoparticle formation.

14. The composition of any one of claims 1-10, wherein the nanoparticles have an average diameter of about 1000 nm or less for at least about 2 hours after nanoparticle formation.

15. The composition of any one of claims 1-10, wherein the nanoparticles have an average diameter of about 10 nm or greater for at least about 2 hours nanoparticle formation.

16. The composition of any one of claims 1-10, the nanoparticles have an average diameter of from about 10 nm to about 1000 nm for at least about 2 hours after nanoparticle formation.

17. The composition of any one of claims 1-16, wherein the nanoparticles have an average diameter of from about 10 nm to about 1000 nm.

18. The composition of claim 17, wherein the nanoparticles have an average diameter of from about 30 nm to about 250 nm.

19. The composition of any one of claims 1-18, wherein the albumin is human serum albumin.

20. The composition of any one of claims 1-19, wherein the molar ratio of the compound of Formula (I) to the pharmaceutically acceptable carrier is from about 1:1 to about 20:1.

21. The composition of claim 20, wherein the molar ratio of the compound of Formula (I) to the pharmaceutically acceptable carrier is from about 2:1 to about 12:1.

22. The composition of any one of claims 1-21, wherein the nanoparticles are suspended, dissolved, or emulsified in a liquid.

23. The composition of any one of claims 1-22, wherein the composition is sterile filterable.

24. The composition of any one of claims 1-23, wherein the composition is dehydrated.

25. The composition of claim 24, wherein the composition is a lyophilized composition.

26. The composition of claim 24 or 25, wherein the composition comprises from about 0.9% to about 24% by weight of the compound of Formula (I), or a pharmaceutically acceptable salt thereof.

27. The composition of claim 26, wherein the composition comprises from about 1.8% to about 16% by weight of the compound of Formula (I), or a pharmaceutically acceptable salt thereof.

28. The composition of any one of claims 24-27, wherein the composition comprises from about 76% to about 99% by weight of the pharmaceutically acceptable carrier.

29. The composition of claim 28, wherein the composition comprises from about 84% to about 98% by weight of the pharmaceutically acceptable carrier.

30. The composition of any one of claims 24-29, wherein the composition is reconstituted with an appropriate biocompatible liquid to provide a reconstituted composition.

31. The composition of claim 30, wherein the appropriate biocompatible liquid is a buffered solution.

32. The composition of claim 30, wherein the appropriate biocompatible liquid is a solution comprising dextrose.

33. The composition of claim 30, wherein the appropriate biocompatible liquid is a solution comprising one or more salts.

34. The composition of claim 30, wherein the appropriate biocompatible liquid is sterile water, saline, phosphate-buffered saline, 5% dextrose in water solution, Ringer's solution, or Ringer's lactate solution.

35. The composition of any one of claims 30-34, wherein the nanoparticles have an average diameter of from about 10 nm to about 1000 nm after reconstitution.

36. The composition of claim 35, wherein the nanoparticles have an average diameter of from about 30 nm to about 250 nm after reconstitution.

37. The composition of any one of claims 1-36, wherein the composition is suitable for injection.

38. The composition of any one of claims 1-37, wherein the composition is suitable for intravenous administration.

39. The composition of any one of claims 1-36, wherein the composition is administered intraperitoneally, intraarterially, intrapulmonarily, orally, by inhalation, intravesicularly, intramuscularly, intratracheally, subcutaneously, intraocularly, intrathecally, intratumorally, or transdermally.

40. A method of treating a disease in a subject in need thereof comprising administering the composition of any one of claims 1-39.

41. A process of preparing a composition of any one of claims 1-39 comprising
a) dissolving a compound of Formula (I) in a volatile solvent to form a solution comprising a dissolved compound of Formula (I);
b) adding the solution comprising the dissolved compound of Formula (I) to a pharmaceutically acceptable carrier in an aqueous solution to form an emulsion;
c) subjecting the emulsion to homogenization to form a homogenized emulsion; and
d) subjecting the homogenized emulsion to evaporation of the volatile solvent to form the composition of any one of claims 1-39.

42. The process of claim 41, wherein the volatile solvent is a chlorinated solvent, alcohol, ketone, ester, ether, acetonitrile, or any combination thereof.

43. The process of claim 42, wherein the volatile solvent is chloroform, ethanol, methanol, or butanol.

44. The process of any one of claims 41-43, wherein the homogenization is high pressure homogenization.

45. The process of claim 44, wherein the emulsion is cycled through high pressure homogenization for an appropriate amount of cycles.

46. The process of claim 45, wherein the appropriate amount of cycles is from about 2 to about 10 cycles.

47. The process of any one of claims 41-46, wherein the evaporation is accomplished with a rotary evaporator.

48. The process of any one of claims 41-47, wherein the evaporation is under reduced pressure.
